(19) United States
(12) Patent Application Publication
(10) Pub. No.: US 2025/0237787 A1
(43) Pub. Date: Jul. 24, 2025

(54) SILICONE BIOMEDICAL DEVICE WITH POLYMERIZABLE GLYCOSAMINOGLYCANS

(71) Applicant: Bausch + Lomb Ireland Limited, Dublin (IE)

(72) Inventors: James Anthony DiBella, JR., Penfield, NY (US); Wendy G. Ahearn, Rochester, NY (US); Dawn O'Sullivan, Waterford (IE); Róisín O'Brien, Waterford (IE); Sandra Long, Wexford (IE)

(21) Appl. No.: 19/020,729

(22) Filed: Jan. 14, 2025

Related U.S. Application Data
(60) Provisional application No. 63/622,213, filed on Jan. 18, 2024.

(51) Int. Cl. G02B 1/04

(52) U.S. Cl. CPC G02B 1/043

(57) ABSTRACT

A silicone biomedical device is disclosed. The silicone biomedical device includes a polymerization product of a biomedical device-forming mixture containing (a) one or more grafted glycosaminoglycan polymers including a glycosaminoglycan having a polymer backbone and one or more side chains comprising an ethylenically unsaturated reactive-containing residue grafted onto the polymer backbone, and (b) one or more silicone biomedical device-forming monomers comprising at least one hydrogen donating group.

### PRIORITY CLAIM

The present application claims priority to U.S. Provisional Patent Application Ser. No. 63/622,213, entitled “Silicone Biomedical Device with Polymerizable Glycosaminoglycans,” filed Jan. 18, 2024, the content of which is incorporated by reference herein in its entirety.

### BACKGROUND

It is highly desirable that a contact lens be as comfortable as possible for wearers. Manufacturers of contact lenses are continually working to improve the comfort of the lenses. Nevertheless, many people who wear contact lenses still experience dryness or eye irritation throughout the day and particularly towards the end of the day. An insufficiently wetted lens at any point in time will cause significant discomfort to the lens wearer. Although wetting drops can be used as needed to alleviate such discomfort, it would certainly be desirable if such discomfort did not arise in the first place.

Contact lenses made from silicone-containing materials have been investigated for a number of years. Such materials can generally be subdivided into two major classes: hydrogels and non-hydrogels. Non-hydrogels do not absorb appreciable amounts of water, whereas hydrogels can absorb and retain water in an equilibrium state. Hydrogels generally have a water content greater than about five wt. % and more commonly between about 10 wt. % to about 80 wt. %. Regardless of their water content, both non-hydrogel and hydrogel silicone contact lenses tend to have relatively hydrophobic, non-wettable surfaces.

Glycosaminoglycans (GAGs) are a group of polysaccharides built of repeating disaccharide units. Due to high polarity and water affinity, they can be found in many systems of human and animal bodies. For example, GAGs occur on the surface of cells and in the extracellular matrix of animal organisms such as skin, cartilage, and lungs.

GAGs each have a chemical structure including a repeating basal disaccharide structure consisting of uronic acid and hexosamine and being optionally sulfated to various degrees. GAGs are mainly classified, depending on the disaccharides constituting them, into three groups: a first group of compounds composed of chondroitin sulfate or dermatan sulfate, a second group of compounds composed of heparan sulfate or heparin, and a third group of hyaluronic acid compounds. For example, the compounds composed of chondroitin sulfate or dermatan sulfate consist of a disaccharide:uronic acid (glucuronic acid or iduronic acid) (β1→3) N-acetylgalactosamine, the compounds composed of heparan sulfate or heparin consist of a disaccharide:uronic acid (glucuronic acid or iduronic acid) (β1→4)N-acetylglucosamine, and the hyaluronic acid consists of a disaccharide:glucuronic acid (β1→3)N-acetylglucosamine. In addition, the structure is highly diverse due to a combination with modification by sulfation.

These GAGs are known as biological materials having both physicochemical properties derived from characteristic viscoelasticity and biological properties mediated by interactions with various functional proteins, depending on the molecular size and the sulfation pattern.

### SUMMARY

In accordance with an illustrative embodiment, a silicone biomedical device comprises a polymerization product of a silicone biomedical device-forming mixture comprising:
(a) one or more grafted glycosaminoglycan polymers comprising a glycosaminoglycan having a polymer backbone and one or more side chains comprising an ethylenically unsaturated reactive-containing residue grafted onto the polymer backbone, and
(b) one or more silicone biomedical device-forming monomers comprising at least one hydrogen donating group.

In accordance with another illustrative embodiment, a method for making a silicone biomedical device comprises:
(a) providing a silicone biomedical device-forming mixture comprising (i) one or more grafted glycosaminoglycan polymers comprising a glycosaminoglycan having a polymer backbone and one or more side chains comprising an ethylenically unsaturated reactive-containing residue grafted onto the polymer backbone, and (ii) one or more silicone biomedical device-forming monomers comprising at least one hydrogen donating group,
(b) subjecting the mixture to polymerization conditions to provide a polymerized silicone biomedical device, and
(c) hydrating the polymerized silicone biomedical device.

### DETAILED DESCRIPTION

Various non-limiting illustrative embodiments described herein are directed to silicone biomedical devices obtained from one or more grafted glycosaminoglycan polymers comprising a glycosaminoglycan having a polymer backbone and one or more side chains comprising an ethylenically unsaturated reactive-containing residue grafted onto the polymer backbone which are polymerized with one or more silicone biomedical device-forming monomers comprising at least one hydrogen donating group.

In the field of biomedical devices such as contact lenses, various physical and chemical properties such as, for example, oxygen permeability, wettability, material strength and stability, are but a few of the factors that must be carefully balanced in order to provide a useable contact lens. For example, since the cornea receives its oxygen supply from contact with the atmosphere, good oxygen permeability is an important characteristic for certain contact lens materials. Wettability also is important in that, if the lens is not sufficiently wettable, it does not remain lubricated and therefore cannot be worn comfortably in the eye. Accordingly, the optimum contact lens would have at least both excellent oxygen permeability and excellent tear fluid wettability.

Although lenses with high-water content are softer, more lubricious and more comfortable to wear, such lenses may not have one or more properties useful to provide comfortable and safe wearing of the contact lenses. Hydrogels represent a desirable class of materials for many biomedical applications, including contact lenses and intraocular lenses. Hydrogels are hydrated, crosslinked polymeric systems that contain water in an equilibrium state. Silicone hydrogels are a known class of hydrogels and are characterized by the inclusion of a siloxy-containing material. An advantage of silicone hydrogels over non-silicone hydrogels is that the silicone hydrogels typically have higher oxygen permeability due to the inclusion of the siloxy-containing monomer. For example, the presently available non-silicone hydrogels shown below in Table 1 have the following water content and oxygen permeability (Dk).

TABLE 1

| Lens | Water Content | Dk |
|---|---|---|
| polymacon | 38 | 8 |
| hilafilcon B | 59 | 22 |
| alphafilcon A | 66 | 32 |
| nelfilcon A | 69 | 26 |
| nesofilcon A | 78 | 42 |

In addition, a problem associated with glycosaminoglycans is that they are known to be incompatible when combined with a silicone monomer in water.

The silicone biomedical devices described in exemplary embodiments herein overcome the foregoing drawbacks described above and advantageously contain a high-water content and are optically clear. For example, the silicone biomedical devices described herein overcome the foregoing drawbacks by being formed from a polymerization product of a silicone biomedical device-forming mixture include one or more grafted glycosaminoglycan polymers comprising a glycosaminoglycan having a polymer backbone and one or more side chains comprising an ethylenically unsaturated reactive-containing residue grafted onto the polymer backbone and one or more silicone biomedical device-forming monomers comprising at least one hydrogen donating group which are optically clear as compared to silicone biomedical devices obtained from a polymerization product of mixture including one or more grafted glycosaminoglycan polymers comprising a glycosaminoglycan having a polymer backbone and one or more side chains comprising an ethylenically unsaturated reactive-containing residue grafted onto the polymer backbone and one or more silicone biomedical device-forming monomers comprising no hydrogen donating groups.

Thus, the silicone biomedical devices disclosed herein will exhibit both suitable physical and chemical properties, e.g., oxygen permeability, lubriciousness and wettability, for prolonged contact with the body. In addition, the grafted glycosaminoglycan polymers employed in making the silicone biomedical devices are believed to advantageously exhibit less enzymatic, oxidative and thermal degradation and thus higher stability, longer shelf life and rigidity of desired conformation.

The silicone biomedical devices disclosed herein are intended for direct contact with body tissue or body fluid. The term “biomedical device” as used herein is any article that is designed to be used while either in or on mammalian tissues or fluid, and preferably in or on human tissue or fluids. Representative examples of biomedical devices include, but are not limited to, artificial ureters, diaphragms, intrauterine devices, heart valves, catheters, denture liners, prosthetic devices, and ophthalmic lens applications, where the lens is intended for direct placement in or on the eye, such as, for example, intraocular devices and contact lenses. In one illustrative embodiment, a silicone biomedical device is a silicone ophthalmic device, particularly a silicone contact lens, and more particularly a silicone contact lens made from silicone hydrogels.

As used herein, the term “ophthalmic device” refers to devices that reside in or on the eye. These devices can provide optical correction, wound care, drug delivery, diagnostic functionality or cosmetic enhancement or effect or a combination of these properties. Useful ophthalmic devices include, but are not limited to, ophthalmic lenses such as soft contact lenses, e.g., a soft, hydrogel lens, soft, non-hydrogel lens and the like, hard contact lenses, e.g., a hard, gas permeable lens material and the like, intraocular lenses, overlay lenses, ocular inserts, optical inserts and the like. As is understood by one skilled in the art, a lens is considered to be “soft” if it can be folded back upon itself without breaking.

In a non-limiting illustrative embodiment, a silicone biomedical device disclosed herein will be a high-water content silicone biomedical device. For example, in an illustrative embodiment, a high-water content silicone biomedical device will have an equilibrium water content of at least about 70 wt. %. In another illustrative embodiment, a high-water content silicone biomedical device will have an equilibrium water content of from about 70 wt. % to about 90 wt. %. In another illustrative embodiment, a high-water content silicone biomedical device will have an equilibrium water content of at least about 75 wt. %. In another illustrative embodiment, a high-water content silicone biomedical device will have an equilibrium water content of from about 75 wt. % to about 90 wt. %.

In non-limiting illustrative embodiments, a silicone biomedical device disclosed herein is formed from a polymerization product of a silicone biomedical device-forming mixture comprising (a) one or more grafted glycosaminoglycan polymers comprising a glycosaminoglycan having a polymer backbone and one or more side chains comprising an ethylenically unsaturated reactive residue grafted onto the polymer backbone, and (b) one or more silicone biomedical device-forming monomers comprising at least one hydrogen donating group.

A glycosaminoglycan (GAG) is one molecule with many alternating subunits. In general, GAGs are represented by the formula A-B-A-B-A-B, where A is a uronic acid and B is an amino sugar that may or may not be either O- or N-sulfated, where the A and B units can be heterogeneous with respect to epimeric content or sulfation. Any natural or synthetic polymer containing uronic acid can be used. Other GAGs are sulfated at different sugars. There are many different types of GAGs having commonly understood structures such as, for example, chondroitin sulfate (e.g., chondroitin 4- and 6-sulfates), heparan, heparin sulfate, heparosan, dermatan, dermatan sulfate, hyaluronic acid or a salt thereof, e.g., sodium hyaluronate or potassium hyaluronate, keratan sulfate, and other disaccharides such as sucrose, lactulose, lactose, maltose, trehalose, cellobiose, mannobiose and chitobiose. Glycosaminoglycans can be purchased from Sigma, and many other biochemical suppliers such as HTL Biotechnology (France). For example, in an illustrative embodiment, the GAG is hyaluronic acid or a salt thereof. In another illustrative embodiment, the GAG is chondroitin sulfate.

The GAGs will have a reactive functional group in the polymer backbone for grafting the ethylenically unsaturated reactive residue onto the backbone. Suitable reactive functional groups in the polymer backbone include carboxylate-containing groups, hydroxyl-containing groups, silicone hydride groups, sulfur-containing groups such as thiols and other groups including polymerizable functionalities such as allylic, vinylic, acrylate, methacylate, methacrylamide etc. In addition, the sugar rings of the GAGs can be opened to form aldehydes for further functionalization. The GAGs for use herein can have a weight average molecular weight ranging from about 10,000 to about 3,000,000 Daltons (Da) in which the lower limit is from about 10,000, about 20,000, about 30,000, about 40,000, about 50,000, about 60,000, about 70,000, about 80,000, about 90,000, or about 100,000, and the upper limit is about 200,000, about 300,000, about 400,000, about 500,000, about 600,000, about 700,000, about 800,000, about 900,000, about 1,000,000, or about up to 2,800,000 Da, where any of the lower limits can be combined with any of the upper limits.

Hyaluronic acid is a well-known, naturally occurring, water soluble biodegradable polymer composed of two alternatively linked sugars, D-glucuronic acid and N-acetylglucosamine, linked via alternating β-(1,4) and β-(1,3) glycosidic bonds. Hyaluronic acid is a non-sulfated GAG. The polymer is hydrophilic and highly viscous in an aqueous solution at relatively low solute concentrations. It often occurs naturally as the sodium salt, sodium hyaluronate. However, other salts are contemplated herein such as, for example, potassium hyaluronate. Methods of preparing commercially available hyaluronan and salts thereof are well known. Hyaluronan can be purchased from Seikagaku Company, Clear Solutions Biotech, Inc., Pharmacia Inc., Sigma Inc., and many other suppliers HTL Biotechnology, Contipro and Bloomage Biotechnology Corporation. Hyaluronic acid has repeating units of the structure represented by the following formula:

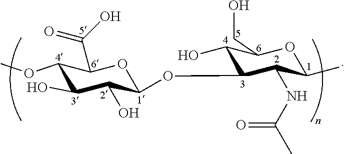

Accordingly, the repeating units in hyaluronic acid can be as follows:

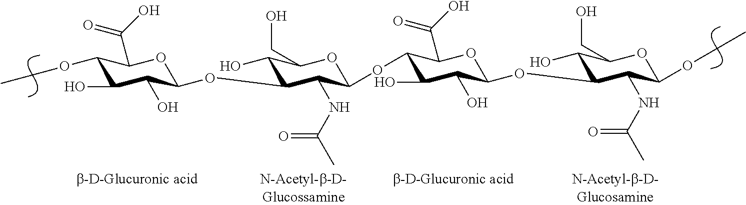

In general, hyaluronic acid or a salt thereof can have from about 2 to about 1,500,000 disaccharide units. In one embodiment, hyaluronic acid or a salt thereof can have a weight average molecular weight ranging from about 10,000 to about 3,000,000 Da in which the lower limit is from about 10,000, about 20,000, about 30,000, about 40,000, about 50,000, about 60,000, about 70,000, about 80,000, about 90,000, or about 100,000, and the upper limit is about 200,000, about 300,000, about 400,000, about 500,000, about 600,000, about 700,000, about 800,000, about 900,000, about 1,000,000, or about up to 2,800,000 Da, where any of the lower limits can be combined with any of the upper limits.

Chondroitin sulfate is a linear sulfated polysaccharide composed of repeating (3-D-glucuronic acid (GlcA) and N-acetyl-β-D-galactosamine (GalNAc) units arranged in the sequence by GlcA-β(1,3)-GalNAc-β(1,4) glycosidic bonds. In one embodiment, chondroitin sulfate has one or more repeating units of the structure represented by the following formula:

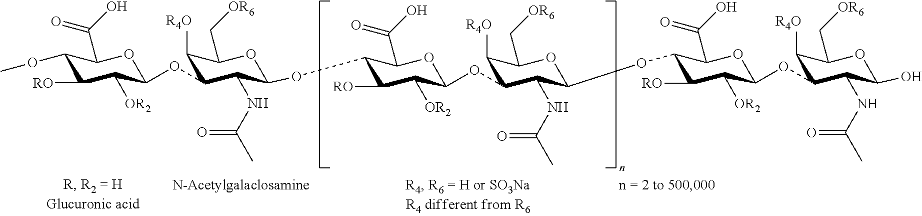

In an illustrative embodiment, chondroitin sulfate has repeating units of the structure represented by the following formula:

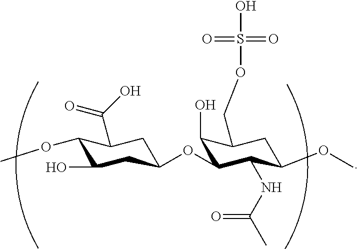

In general, chondroitin sulfate can have from about 2 to about 1,500,000 repeating units. In one embodiment, chondroitin sulfate can have a weight average molecular weight ranging from about 10,000 to about 3,000,000 Da in which the lower limit is from about 5,000, 10,000, about 20,000, about 30,000, about 40,000, about 50,000, about 60,000, about 70,000, about 80,000, about 90,000, or about 100,000, and the upper limit is about 200,000, about 300,000, about 400,000, about 500,000, about 600,000, about 700,000, about 800,000, about 900,000, about 1,000,000, or about 3,000,000 Da, where any of the lower limits can be combined with any of the upper limits.

In an illustrative embodiment, dermatan sulfate has repeating units of the structure represented by the following formula:

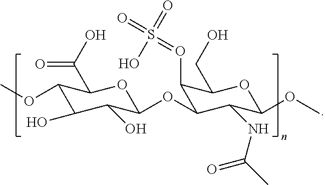

In general, dermatan sulfate can have from about 2 to about 1,500,000 repeating units. In one embodiment, chondroitin sulfate can have a weight average molecular weight ranging from about 10,000 to about 3,000,000 Da in which the lower limit is from about 5,000, 10,000, about 20,000, about 30,000, about 40,000, about 50,000, about 60,000, about 70,000, about 80,000, about 90,000, or about 100,000, and the upper limit is about 200,000, about 300,000, about 400,000, about 500,000, about 600,000, about 700,000, about 800,000, about 900,000, about 1,000,000, or about 3,000,000 Da, where any of the lower limits can be combined with any of the upper limits.

In an illustrative embodiment, heparin and heparin sulfate has repeating units of the structure represented by the following formula:

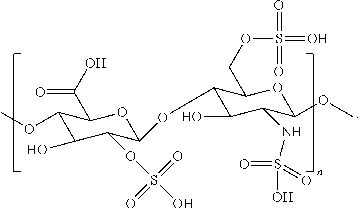

In general, heparin and heparin sulfate can have from about 2 to about 1,500,000 repeating units. In one embodiment, chondroitin sulfate can have a weight average molecular weight ranging from about 10,000 to about 3,000,000 Da in which the lower limit is from about 5,000, 10,000, about 20,000, about 30,000, about 40,000, about 50,000, about 60,000, about 70,000, about 80,000, about 90,000, or about 100,000, and the upper limit is about 200,000, about 300,000, about 400,000, about 500,000, about 600,000, about 700,000, about 800,000, about 900,000, about 1,000,000, or about 3,000,000 Da, where any of the lower limits can be combined with any of the upper limits.

In an illustrative embodiment, keratan sulfate has repeating units of the structure represented by the following formula:

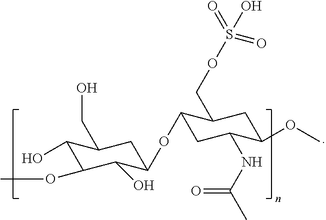

In general, keratan sulfate can have from about 2 to about 1,500,000 repeating units. In one embodiment, chondroitin sulfate can have a weight average molecular weight ranging from about 10,000 to about 3,000,000 Da in which the lower limit is from about 5,000, 10,000, about 20,000, about 30,000, about 40,000, about 50,000, about 60,000, about 70,000, about 80,000, about 90,000, or about 100,000, and the upper limit is about 200,000, about 300,000, about 400,000, about 500,000, about 600,000, about 700,000, about 800,000, about 900,000, about 1,000,000, or about 3,000,000, where any of the lower limits can be combined with any of the upper limits.

The ethylenically unsaturated reactive-containing residue grafted onto a reactive functional group in the polymer backbone of the GAG is derived from a monomer comprising an ethylenically unsaturated reactive group and at least one reactive end group. In one embodiment, the ethylenically unsaturated reactive-containing residue is a methacrylate-containing residue. The at least one reactive end group includes a reactive functional group capable of grafting on to a complementary reactive functional group in the polymer backbone of the GAG. Suitable reactive functional groups of the monomer comprising an ethylenically unsaturated reactive group and at least one reactive end group include, for example, a halogen, an anhydride, an amino group, an aldehyde group, a carboxylic acid group, an alcohol group, a thiol group, a hydrazide group, a glycidyl group, etc. In one non-limiting illustrative embodiment, an ethylenically unsaturated reactive-containing residue can be derived from, for example, methacrylic anhydride, methacryloyl chloride, 2-isocyanoethylmethacrylate, 3-(trimethoxysilyl)propyl methacrylate, 3-(chlorodimethylsilyl)propyl methacrylate, glycidyl methacrylate, methacryloyl hydrazide, aminoethyl methacrylate, vinyl chloroformate, allyl chloride, acryloyl chloride, and acrylic anhydride. However, the foregoing list is merely exemplary and other monomers for forming the ethylenically unsaturated reactive-containing residue on the polymer backbone of the GAG are contemplated herein.

The grafted glycosaminoglycan polymers disclosed herein can be obtained by grafting the at least one reactive end group of the one or more monomers comprising an ethylenically unsaturated reactive group onto a complementary reactive functionality in the polymer backbone of the glycosaminoglycan. For example, in one illustrative embodiment, an anhydride group of the one or more monomers comprising an ethylenically unsaturated reactive group can be grafted onto a carboxylic acid group in the polymer backbone of the glycosaminoglycan. In non-limiting illustrative embodiments, the graft polymerization reaction can obtain a degree of grafting, i.e., the number of sidechains in the polymer backbone containing the ethylenically unsaturated reactive-containing residue, ranging from about 0.5% to about 50%. In another illustrative embodiment, the degree of grafting can range from about 2% to about 30%. In another illustrative embodiment, the degree of grafting can range from about 5% to about 20%. In yet another illustrative embodiment, the degree of grafting can range from about 5% to about 15%. In yet another illustrative embodiment, the degree of grafting can range from about 5% to about 10%.

In general, the GAG and monomer comprising an ethylenically unsaturated reactive group and at least one reactive end group can be added sequentially or simultaneously to a reaction mixture. The reaction can be carried out at a suitable temperature and for a time period for the completion of the reaction to maximize the yield of the product ethylenically unsaturated reactive-containing residue grafted onto the polymer backbone of the glycosaminoglycan. For example, in an illustrative embodiment, a suitable temperature and time period includes a temperature ranging from about 10° C. to about 40° C. and a time period ranging from about 4 hours to about 48 hours. In an illustrative embodiment, a suitable temperature and time period includes a temperature ranging from about 15° C. to about 25° C. and a time period ranging from about 8 hours to about 24 hours.

In an illustrative embodiment, a glycosaminoglycan can be added to the reaction mixture in an amount ranging from about 0.5 wt. % to about 5 wt. %, based on the total weight of the reaction mixture. In one illustrative embodiment, a glycosaminoglycan can be added to the reaction mixture in an amount ranging from about 1 wt. % to about 3 wt. %, based on the total weight of the reaction mixture.

In an illustrative embodiment, a monomer comprising an ethylenically unsaturated reactive group and at least one reactive end group can be added to the reaction mixture in an amount ranging from about 0.1 wt. % to about 5 wt. %, based on the total weight of the reaction mixture. In one illustrative embodiment, a monomer comprising an ethylenically unsaturated reactive group and at least one reactive end group can be added to the reaction mixture in an amount ranging from about 0.5 wt. % to about 2 wt. %, based on the total weight of the reaction mixture.

In a non-limiting illustrative embodiment, the ethylenically unsaturated reactive-containing residue is a methacrylate-containing residue derived from a methacrylate-containing monomer as described above, and the degree of methacrylation can range from about 0.5% to about 50%. In another illustrative embodiment, the degree of methacrylation can range from about 2% to about 30%. In yet another illustrative embodiment, the degree of methacrylation can range from about 5% to about 15%.

The resulting grafted glycosaminoglycan polymer can be a random copolymer or a block copolymer. In one illustrative embodiment, a grafted glycosaminoglycan polymer disclosed herein can have a weight average molecular weight ranging from about 20,000 to about 6,000,000 Da in which the lower limit is from about 20,000, about 30,000, about 40,000, about 50,000, about 60,000, about 70,000, about 80,000, about 90,000, or about 100,000 Da, and the upper limit is about 100,000, about 150,000, about 200,000, about 300,000, about 400,000, about 500,000, about 600,000, about 700,000, about 800,000, about 900,000, about 1,000,000, about 2,000,000, about 3,000,000, about 4,000,000, about 5,000,000 or up to about 6,000,000 Da, wherein any of the lower limits can be combined with any of the upper limits.

In non-limiting illustrative embodiments, as may be combined with one or more of the preceding paragraphs, the grafted glycosaminoglycan polymer can be present in the silicone biomedical device-forming mixture in an amount ranging from about 0.1 wt. % to about 1.0 wt. %, based on the total weight of the silicone biomedical device-forming mixture. In another embodiment, the grafted glycosaminoglycan polymer can be present in the silicone biomedical device-forming mixture in an amount ranging from about 0.25 wt. % to about 0.5 wt. %, based on the total weight of the silicone biomedical device-forming mixture.

In non-limiting illustrative embodiments, as may be combined with one or more of the preceding paragraphs, the silicone biomedical device-forming mixture described herein further includes one or more silicone biomedical device-forming monomers comprising at least one hydrogen donating group.

The term “hydrogen donating group” as used herein refers to a hydrogen atom that is able to form reversible (inter- or intramolecular) physical interactions with another atom or group. In an illustrative embodiment, suitable hydrogen donating groups include, for example, a hydroxy (OH) group, an amino (NH, NH₂) group, a SH group, a carboxyl group, e.g., COOH, COSH. In an illustrative embodiment, a hydrogen donating group is OH or NH. However, these are merely illustrative and any hydrogen donating group is contemplated herein.

The silicone biomedical device-forming monomers comprising at least one hydrogen donating group will have a suitable ratio of silicon atoms to hydrogen donating groups to provide the desired degree of compatibilization. In non-limiting illustrative embodiments, as may be combined with one or more of the preceding paragraphs, the silicone biomedical device-forming monomers comprising at least one hydrogen donating group can have a ratio of silicon atoms to hydrogen donating groups of from about 1:1 to about 9:1. In non-limiting illustrative embodiments, the silicone biomedical device-forming monomers comprising at least one hydrogen donating group can have a ratio of silicon atoms to hydrogen donating groups of from about 1:1 to about 4:1. In non-limiting illustrative embodiments, the silicone biomedical device-forming monomers comprising at least one hydrogen donating group can have a ratio of silicon atoms to hydrogen donating groups of from about 1:1 to about 1:5.

In addition to the at least one hydrogen donating group, the silicone biomedical device-forming monomers will further contain at least one ethylenically unsaturated reactive group for polymerizing with the ethylenically unsaturated reactive group of the grafted glycosaminoglycan polymer. Ethylenically unsaturated reactive end groups are well known to those skilled in the art. Suitable ethylenically unsaturated polymerizable groups include, for example, (meth)acrylates, vinyl carbonates, O-vinyl carbamates, N-vinyl carbamates, styrene-containing radicals and (meth)acrylamides. As used herein, the term “(meth)” denotes an optional methyl substituent. Thus, terms such as “(meth)acrylate” denote either methacrylate or acrylate, and “(meth)acrylamide” denotes either methacrylamide or acrylamide. In one embodiment, an ethylenically unsaturated reactive end group can be represented by the general formula:

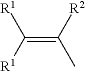

wherein R¹ is independently hydrogen, fluorine or methyl; R² is independently hydrogen, fluorine, an alkyl radical having 1 to 6 carbon atoms, or a —CO—Y—R³ radical wherein Y is —O—, —S— or —NH— and R³ is a divalent alkylene radical having 1 to about 10 carbon atoms.

In another illustrative embodiment, the (meth)acrylic group of a heterocyclic (meth)acrylic monomer is a (meth)acrylate-containing reactive end group. Suitable (meth)acrylate-containing reactive end groups can be those represented by the structure:

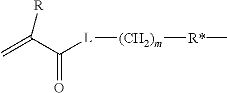

wherein R is hydrogen or methyl; L is O, NR¹, or S, where R¹ is H, CH₃, CH₂CH₃, or CH(CH₃)₂; m is an integer from 0 to 4 and R* is a linking group or bond. Suitable linking groups include, for example, any divalent hydrocarbon radical or moiety such as independently straight or branched, substituted or unsubstituted C₁-C₆ alkyl group, or an —OR² group where R² is an alkyl group from 1 to 6 carbon atoms.

In some embodiments, the silicone biomedical device-forming monomer is a hydroxyl-functionalized silicone biomedical device-forming monomer. In non-limiting illustrative embodiments, a class of hydroxyl-functionalized silicone biomedical device-forming monomers include monomers of Formulae I and II:

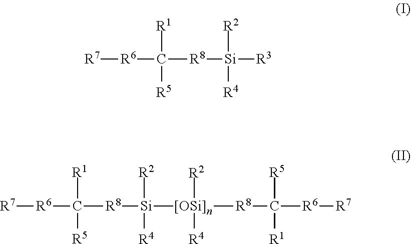

wherein:
n is an integer between 3 and 35, or between 4 and 25;
R¹ is hydrogen or C₁₋₆ alkyl;
R², R³, and R⁴, are independently, substituted or unsubstituted C₁₋₆ alkyl, C₁₋₆ triC₁₋₆ alkylsiloxy, phenyl, naphthyl, substituted phenyl, or substituted naphthyl where the alkyl substitutents include, for example, C₁₋₆ alkoxycarbonyl, C₁₋₆ alkyl, C₁₋₆ alkoxy, amide, halogen, hydroxyl, carboxyl, C₁₋₆ alkylcarbonyl and formyl, and where the aromatic substitutents include, for example, C₁₋₆ alkoxycarbonyl, C₁₋₆ alkyl, C₁₋₆ alkoxy, amide, halogen, hydroxyl, carboxyl, C₁₋₆ alkylcarbonyl and formyl;
R⁵ is hydroxyl, an alkyl group containing one or more hydroxyl groups; or (CH₂(CR⁹R¹⁰)_{y}O)ₓ)—R¹¹ wherein y is 1 to 5, or 1 to 3, x is an integer of 1 to 100, preferably 2 to 90 and more preferably 10 to 25; R⁹-R¹¹ are independently selected from H, alkyl having up to 10 carbon atoms and alkyls having up to 10 carbon atoms substituted with at least one polar functional group;
R⁶ is a divalent group comprising up to 20 carbon atoms;
R⁷ is a free radical reactive group;
R⁸ is a divalent or trivalent group comprising up to 20 carbon atoms.

In an illustrative embodiment, for monofunctional hydroxyl functionalized silicone containing monomers, R¹ is hydrogen, and R², R³, and R⁴ are C₁₋₆ alkyl and triC₁₋₆ alkylsiloxy, such as methyl and trimethylsiloxy. In an illustrative embodiment, for multifunctional (difunctional or higher) R¹-R⁴ independently comprise ethylenically unsaturated polymerizable groups such as an acrylate, a styryl, a C₁₋₆ alkylacrylate, acrylamide, C₁₋₆ alkylacrylamide, N-vinyllactam, N-vinylamide, C₂₋₁₂ alkenyl, C₂₋₁₂ alkenylphenyl, C₂₋₁₂ alkenylnaphthyl, or C₂₋₆ alkenylphenyl C₁₋₆ alkyl.

In some embodiments, R⁵ is hydroxyl, —CH2OH, —CH2CHOHCH2OH, or a —(O CH₂CH₂)ₓOH with hydroxyl being most preferred.

In some embodiments, R⁶ is a divalent substituted or unsubstituted C₁₋₆ alkyl, substituted or unsubstituted C₁₋₆ alkyloxy, substituted or unsubstituted C₁₋₆ alkyloxy C₁₋₆ alkyl, substituted or unsubstituted phenylene, substituted or unsubstituted naphthalene, substituted or unsubstituted C₁₋₁₂ cycloalkyl, substituted or unsubstituted C₁₋₆ alkoxycarbonyl, substituted or unsubstituted amide, substituted or unsubstituted carboxy, substituted or unsubstituted C₁₋₆ alkylcarbonyl, carbonyl, and substituted or unsubstituted C₁₋₆ alkoxy, where the substituents include, for example, C₁₋₆ alkoxycarbonyl, C₁₋₆ alkyl, C₁₋₆ alkoxy, amide, halogen, hydroxyl, carboxyl, C₁₋₆ alkylcarbonyl and formyl. In some embodiments, R⁶ is a divalent methyl (methylene).

In some embodiments, R⁷ comprises a free radical reactive group, such as an acrylate, a styryl, vinyl, vinyl ether, itaconate group, a C₁₋₆ alkylacrylate, acrylamide, C₁₋₆ alkylacrylamide, N-vinyllactam, N-vinylamide, C₂₋₁₂ alkenyl, C₂₋₁₂ alkenylphenyl, C₂₋₁₂ alkenylnaphthyl, or C₂₋₆ alkenylphenylC₁₋₆ alkyl or a cationic reactive group such as vinyl ether or epoxide groups. In some embodiments, R⁷ comprises a methacrylate.

In some embodiments, R¹ is a divalent substituted or unsubstituted C₁₋₆ alkyl, substituted or unsubstituted C₁₋₆ alkyloxy, substituted or unsubstituted C₁₋₆ alkyloxy C₁₋₆ alkyl, substituted or unsubstituted phenylene, substituted or unsubstituted naphthalene, substituted or unsubstituted C₁₋₁₂ cycloalkyl, substituted or unsubstituted C₁₋₆ alkoxycarbonyl, substituted or unsubstituted amide, substituted or unsubstituted carboxy, substituted or unsubstituted C₁₋₆ alkylcarbonyl, carbonyl, substituted or unsubstituted C₁₋₆ alkoxy, where the substituents include, for example, C₁₋₆ alkoxycarbonyl, C₁₋₆ alkyl, C₁₋₆ alkoxy, amide, halogen, hydroxyl, carboxyl, C₁₋₆ alkylcarbonyl and formyl. In some embodiments, R⁸ is a C₁₋₆ alkyloxy C₁₋₆ alkyl.

A representative example of a hydroxyl-functionalized silicone biomedical device-forming monomer of Formula I includes 2-propenoic acid, 2-methyl-2-hydroxy-3-[3-[1,3,3,3-tetramethyl-1-[(trimethylsilyl)oxy]disiloxanyl]propoxy]propyl ester (also referred to as ((3-methacryloxy-2-hydroxypropyloxy)propylbis(trimethylsiloxy)methylsilane) having the following structure:

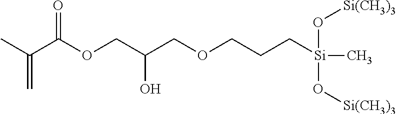

The above compound, (3-methacryloxy-2-hydroxypropyloxy)propylbis(trimethylsiloxy)methylsilane can be formed from an epoxide, which can produce an 80:20 mixture of the compound shown above and (2-methacryloxy-3-hydroxypropyloxy)propylbis(trimethylsiloxy)methylsilane. In some embodiments some amount of the primary hydroxyl is present, such as greater than about 10 wt. % or at least about 20 wt.

In some embodiments, suitable hydroxyl-functionalized silicone biomedical device-forming monomers include (3-methacryloxy-2-hydroxypropyloxy)propyltris(trimethylsiloxy)silane having the following structure:

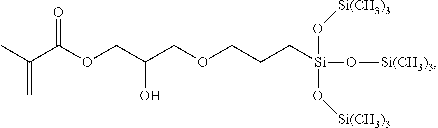

bis-3-methacryloxy-2-hydroxypropyloxypropyl polydimethylsiloxane having the following structure:

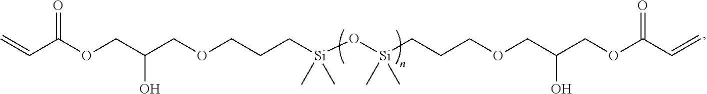

3-methacryloxy-2-(2-hydroxyethoxy)propyloxy)propylbis(trimethylsiloxy)methylsilane having the following structure:

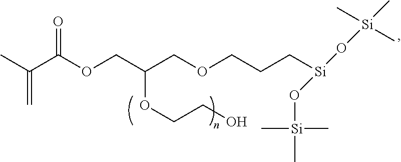

and N,N,N′,N′-tetrakis(3-methacryloxy-2-hydroxypropyl)-α,ω-bis-3-aminopropyl-polydimethylsiloxane.

In some embodiments, suitable hydroxyl-functionalized silicone biomedical device-forming monomers include reaction products of glycidyl methacrylate with amino-functional polydimethylsiloxanes. Other suitable hydroxyl-functionalized silicone biomedical device-forming monomers include those disclosed in columns 6, 7 and 8 of U.S. Pat. No. 5,994,488, and monomers disclosed in U.S. Pat. Nos. 4,235,985; 4,259,467; 4,260,725; 4,261,875; 4,649,184; 4,139,513, and 4,139,692, and U.S. 2002/0016383. These and any other patents or applications cited herein are incorporated by reference.

In some embodiments, suitable hydroxyl-functionalized silicone biomedical device-forming monomers include those having the following structure:

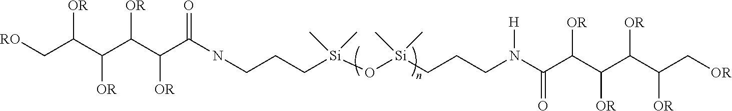

where n=1-50 and R independently comprises H or a polymerizable unsaturated group, with at least one R comprising a polymerizable group, and at least one R, and preferably 3 to 8 R, comprise H.

In some embodiments, another class of hydroxyl-functionalized silicone biomedical device-forming monomers include monomers of Formula III:

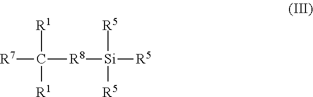

wherein R¹, R⁵, R⁷ and R⁸ have the aforestated meanings, wherein at least one of R⁵ is an OH group. In some embodiments, at least two of R⁵ are an OH group. In some embodiments, each R¹ is hydrogen, each R⁵ is an OH group, R⁷ is a methacrylate end group and R⁸ is a C₁₋₆ alkyl group. A representative example of a monomer of Formula III is 3-(trihydroxysilyl)propyl methacrylate. The monomers of Formula III can be prepared by methods known in the art, and as set forth in the examples.

In some embodiments, the silicone biomedical device-forming monomer is an amino-functionalized silicone biomedical device-forming monomer. In non-limiting illustrative embodiments, a class of amino-functionalized silicone biomedical device-forming monomers include monomers of Formula IV:

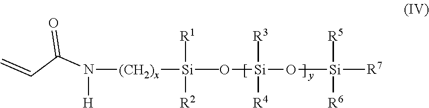

wherein R¹, R², R³ and R⁴ are independently hydrogen, an alkyl group, a halo alkyl group, a cycloalkyl group, a heterocycloalkyl group, an alkenyl group, a haloalkenyl group, an aryl group and a heteroaryl group; R⁵, R⁶ and R⁷ are independently a straight or branched alkyl group; x is from 1 to 6; and y is from 3 to 7.

In some embodiments, R¹, R², R³ and R⁴ of the monofunctional silicone monomer represented by a structure of Formula IV are independently hydrogen, a C₁ to C₁₂ alkyl group, a C₁ to C₁₂ halo alkyl group, a C₃ to C₁₂ cycloalkyl group, a C₃ to C₁₂ heterocycloalkyl group, a C₂ to C₁₂ alkenyl group, a C₂ to C₁₂ haloalkenyl group, a C₆ to C₁₂ aromatic group and a C₆ to C₁₂ heteroaromatic group; R⁵, R⁶ and R⁷ are independently a straight or branched C₁ to C₁₂ alkyl group; x is from 1 to 6; and y is from 3 to 7.

In some embodiments, R¹, R², R³ and R⁴ of the monofunctional silicone monomer represented by a structure of Formula IV are independently hydrogen, a C₁ to C₆ alkyl group; R⁵, R⁶ and R⁷ are independently a straight or branched C₁ to C₆ alkyl group; x is from 1 to 6; and y is from 3 to 7.

In some embodiments, R¹, R², R³ and R⁴ of the monofunctional silicone monomer represented by a structure of Formula IV are independently a C₁ to C₃ alkyl group; R⁵ and R⁶ are independently a C₁ to C₃ alkyl group; R⁷ is a straight or branched C₃ to C₆ alkyl group; x is from 2 to 4; and y is from 3 to 7.

Representative examples of alkyl groups for use herein include, by way of example, a straight or branched alkyl chain radical containing carbon and hydrogen atoms of from 1 to about 30 carbon atoms or from 1 to about 12 carbon atoms or from 1 to about 6 carbon atoms with or without unsaturation, to the rest of the molecule, e.g., methyl, ethyl, n-propyl, 1-methylethyl (isopropyl), n-butyl, n-pentyl, methylene, ethylene, etc., and the like, optionally containing one or more heteroatoms, e.g., O and N, and the like, or one or more halogen atoms, e.g., fluorine, chlorine, bromine, and iodine, to form a halo alkyl group.

Representative examples of cycloalkyl groups for use herein include, by way of example, a substituted or unsubstituted, non-aromatic mono or multicyclic ring system of about 3 to about 30 carbon atoms or from 3 to about 12 carbon atoms or from 3 to about 6 carbon atoms such as, for example, cyclopropyl, cyclobutyl, cyclopentyl, cyclohexyl, perhydronapththyl, adamantyl and norbornyl groups, bridged cyclic groups or sprirobicyclic groups, e.g., spiro-(4, 4)-non-2-yl and the like, optionally containing one or more heteroatoms, e.g., O and N, and the like to form a heterocycloalkyl group.

Representative examples of cycloalkylalkyl groups for use herein include, by way of example, a substituted or unsubstituted, cyclic ring-containing radical containing from about 4 to about 30 carbon atoms or from 3 to about 6 carbon atoms directly attached to the alkyl group which is then attached to the main structure of the monomer at any carbon from the alkyl group that results in the creation of a stable structure such as, for example, cyclopropylmethyl, cyclobutylethyl, cyclopentylethyl and the like, wherein the cyclic ring can optionally contain one or more heteroatoms, e.g., O and N, and the like to form a heterocycloalkylalkyl group.

Representative examples of cycloalkenyl groups for use herein include, by way of example, a substituted or unsubstituted cyclic ring-containing radical containing from about 3 to about 30 carbon atoms or from 3 to about 6 carbon atoms with at least one carbon-carbon double bond such as, for example, cyclopropenyl, cyclobutenyl, cyclopentenyl and the like, wherein the cyclic ring can optionally contain one or more heteroatoms, e.g., O and N, and the like to form a heterocycloalkenyl group.

Representative examples of aryl groups for use herein include, by way of example, a substituted or unsubstituted, monoaromatic or polyaromatic radical containing from about 6 to about 30 carbon atoms or from about 6 to about 12 carbon atoms such as, for example, phenyl, naphthyl, tetrahydronapthyl, indenyl, biphenyl and the like, optionally containing one or more heteroatoms, e.g., O and N, and the like to form a heteroaryl group.

In an illustrative embodiment, the monofunctional silicone monomer represented by the structure of Formula IV is either commercially available from such sources as ShinEtsu or can be made by methods within the purview of one skilled in the art. For example, in an illustrative embodiment, the monofunctional silicone monomer represented by the structure of Formula IV can be prepared according to the following reaction Scheme I.

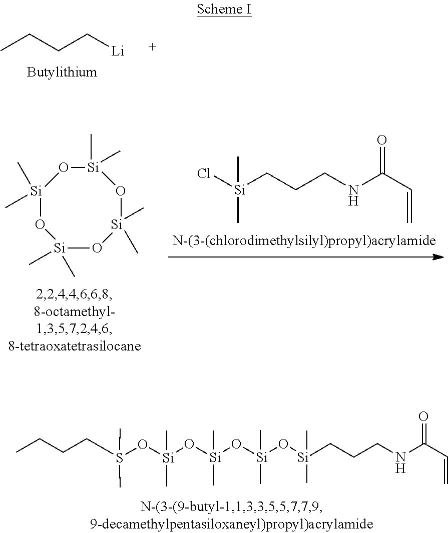

In non-limiting illustrative embodiments, as may be combined with one or more of the preceding paragraphs, the silicone biomedical device-forming monomer can be present in the silicone biomedical device-forming mixture in an amount ranging from about 1 wt. % to about 30 wt. %, based on the total weight of the silicone biomedical device-forming mixture. In another embodiment, the silicone biomedical device-forming monomers can be present in the silicone biomedical device-forming mixture in an amount ranging from about 1 wt. % to about 15 wt. %, based on the total weight of the silicone biomedical device-forming mixture.

In non-limiting illustrative embodiments, as may be combined with one or more of the preceding paragraphs, the silicone biomedical device-forming mixture described herein can further include one or more hydrophilic monomers. Suitable one or more hydrophilic monomers include, for example, unsaturated carboxylic acids, acrylamides, vinyl lactams, poly(alkyleneoxy)(meth)acrylates, hydroxyl-containing-(meth)acrylates, hydrophilic vinyl carbonates, hydrophilic vinyl carbamates, hydrophilic oxazolones, and poly(alkene glycols) functionalized with polymerizable groups and the like and mixtures thereof. Representative examples of unsaturated carboxylic acids include methacrylic acid, acrylic acid and the like and mixtures thereof. Representative examples of amides include alkylamides such as N,N-dimethylacrylamide, N,N-dimethylmethacrylamide and the like and mixtures thereof. Representative examples of cyclic lactams include N-vinyl-2-pyrrolidone, N-vinyl caprolactam, N-vinyl-2-piperidone and the like and mixtures thereof. Representative examples of hydroxyl-containing (meth)acrylates include 2-hydroxyethyl methacrylate, glycerol methacrylate and the like and mixtures thereof. Representative examples of functionalized poly(alkene glycols) include poly(diethylene glycols) of varying chain length containing monomethacrylate or dimethacrylate end caps. In one embodiment, the poly(alkene glycol) polymer contains at least two alkene glycol monomeric units. Still further examples are the hydrophilic vinyl carbonate or vinyl carbamate monomers disclosed in U.S. Pat. No. 5,070,215, and the hydrophilic oxazolone monomers disclosed in U.S. Pat. No. 4,910,277. Other suitable hydrophilic monomers will be apparent to one skilled in the art. Mixtures of the foregoing hydrophilic monomers can also be used in the monomeric mixtures herein.

In non-limiting illustrative embodiments, as may be combined with one or more of the preceding paragraphs, the one or more hydrophilic monomers can be present in the silicone biomedical device-forming mixture in an amount ranging from about 30 wt. % to about 90 wt. %, based on the total weight of the silicone biomedical device-forming mixture. In another illustrative embodiment, the one or more hydrophilic monomers can be present in the silicone biomedical device-forming mixture in an amount ranging from about 45 wt. % to about 75 wt. %, based on the total weight of the silicone biomedical device-forming mixture. In another illustrative embodiment, the one or more hydrophilic monomers can be present in the silicone biomedical device-forming mixture in an amount ranging from greater than or equal to about 50 wt. % to about 75 wt. %, based on the total weight of the silicone biomedical device-forming mixture.

In non-limiting illustrative embodiments, as may be combined with one or more of the preceding paragraphs, the silicone biomedical device-forming mixture can further include one or more hydrophobic monomers. Suitable hydrophobic monomers include ethylenically unsaturated hydrophobic monomers such as, for example, (meth)acrylate-containing hydrophobic monomers, N-alkyl (meth)acrylamide-containing hydrophobic monomers, alkyl vinylcarbonate-containing hydrophobic monomers, alkyl vinylcarbamate-containing hydrophobic monomers, fluoroalkyl (meth)acrylate-containing hydrophobic monomers, N-fluoroalkyl (meth)acrylamide-containing hydrophobic monomers, N-fluoroalkyl vinylcarbonate-containing hydrophobic monomers, N-fluoroalkyl vinylcarbamate-containing hydrophobic monomers, silicone-containing (meth)acrylate-containing hydrophobic monomers, (meth)acrylamide-containing hydrophobic monomers, vinyl carbonate-containing hydrophobic monomers, vinyl carbamate-containing hydrophobic monomers, styrenic-containing hydrophobic monomers, polyoxypropylene (meth)acrylate-containing hydrophobic monomers and the like and mixtures thereof.

In a non-limiting illustrative embodiment, the one or more hydrophobic monomers can be represented by the structure of Formula V:

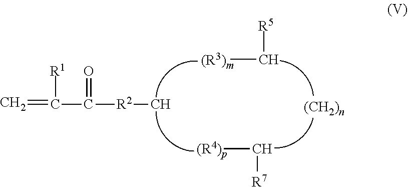

wherein R¹ is methyl or hydrogen; R² is —O— or —NH—; R³ and R⁴ are independently a divalent radical selected from the group consisting of —CH₂—, —CHOH— and —CHR⁶—; R⁵ and R⁶ are independently a branched C₃-C₈ alkyl group; R⁷ is hydrogen or —OH; n is an integer of at least 1, and m and p are independently 0 or an integer of at least 1, provided that the sum of m, p and n is 2, 3, 4 or 5.

Representative examples of one or more hydrophobic monomers represented by the structure of Formula V include, but are not limited to, 4-t-butyl-2-hydroxycyclohexyl methacrylate (TBE); 4-t-butyl-2-hydroxycyclopentyl methacrylate; 4-t-butyl-2-hydroxycyclohexyl methacrylamide (TBA); 6-isopentyl-3-hydroxycyclohexyl methacrylate; 2-isohexyl-5-hydroxycyclopentyl methacrylamide, 4-t-butylcyclohexyl methacrylate, isobornyl methacrylate, adamntyl methacrylate, n-butyl methacrylate, n-hexyl methacrylate, lauryl methacrylate, benzyl methacrylate, and the like. In one embodiment, one or more hydrophobic monomers include compounds of Formula V wherein R³ is —CH₂—, m is 1 or 2, p is 0, and the sum of m and n is 3 or 4.

In non-limiting illustrative embodiments, as may be combined with one or more of the preceding paragraphs, the one or more hydrophobic monomers can be present in the silicone biomedical device-forming mixture in an amount ranging from about 0.5 wt. % to about 25 wt. %, based on the total weight of the silicone biomedical device-forming mixture. In another illustrative embodiment, the one or more hydrophobic monomers will be present in the silicone biomedical device-forming mixture in an amount ranging from about 1 wt. % to about 10 wt. %, based on the total weight of the silicone biomedical device-forming mixture.

In non-limiting illustrative embodiments, as may be combined with one or more of the preceding paragraphs, the silicone biomedical device-forming mixture can further include one or more crosslinking agents. Suitable crosslinking agents for use herein are known in the art. In illustrative embodiments, the one or more crosslinking agents are bi- or polyfunctional crosslinking agents comprising two or more reactive functional groups. In an embodiment, the one or more crosslinking agents have at least two polymerizable functional groups. Representative examples of crosslinking agents include divinylbenzene, allyl methacrylate, ethyleneglycol dimethacrylate, tetraethyleneglycol dimethacrylate, 1,4-butanediol diglycidyl ether, polyethyleneglycol dimethacrylate, vinyl carbonate derivatives of the glycol dimethacrylates, and methacryloxyethyl vinylcarbonate. However, other crosslinking agents are contemplated and the foregoing list is merely exemplary.

In non-limiting illustrative embodiments, as may be combined with one or more of the preceding paragraphs, the one or more crosslinking agents are used in the silicone biomedical device-forming mixture in amounts of less than or equal to about 5 wt. %, and generally less than or equal to about 2 wt. %, e.g., from about 0.1 wt. % to about 5 wt. %, or from about 0.1 wt. % to about 2 wt. %, based on the total weight of the silicone biomedical device-forming mixture.

In non-limiting illustrative embodiments, as may be combined with one or more of the preceding paragraphs, the silicone biomedical device-forming mixture can further include one or more wetting agents. In one embodiment, suitable one or more wetting agents includes, for example, poly(vinyl alcohol) (PVA), poly(N-vinylpyrrolidone) (PVP), polymers containing carboxylic acid functionality, such as a polymer containing poly(acrylic acid) (PAA), copolymers of the foregoing and the like. Another suitable class of wetting agents includes non-polymeric wetting agents. Representative examples of such wetting agents include glycerin, propylene glycol, and other non-polymeric diols and glycols.

In non-limiting illustrative embodiments, as may be combined with one or more of the preceding paragraphs, the one or more wetting agents can be present in the silicone biomedical device-forming mixture in an amount ranging from about 0 wt. % to about 10 wt. %, based on the total weight of the silicone biomedical device-forming mixture. In another embodiment, the one or more wetting agents can be present in the silicone biomedical device-forming mixture in an amount ranging from about 0.1 wt. % to about 10 wt. %, based on the total weight of the silicone biomedical device-forming mixture. In yet another embodiment, the one or more wetting agents can be present in the silicone biomedical device-forming mixture in an amount ranging from about 0.5 wt. % to about 5 wt. %, based on the total weight of the silicone biomedical device-forming mixture.

In non-limiting illustrative embodiments, as may be combined with one or more of the preceding paragraphs, the silicone biomedical device-forming mixture can further include one or more surfactants such as end terminal functionalized surfactants. A suitable end terminal functionalized surfactant includes, by way of example, one or more end terminal functionalized polyethers. Useful polyethers to be end terminal functionalized comprise one or more chains or polymeric components which have one or more (—O—R—) repeat units wherein R is an alkylene or arylene group having 2 to about 6 carbon atoms. The polyethers may be derived from block copolymers formed from different ratio components of ethylene oxide (EO) and propylene oxide (PO). Such polyethers and their respective component segments may include different attached hydrophobic and hydrophilic chemical functional group moieties and segments.

A representative example of a suitable polyether which can be end terminal functionalized is a poloxamer block copolymer. One specific class of poloxamer block copolymers are those available under the trademark Pluronic (BASF Wyandotte Corp., Wyandotte, Mich.). Poloxamers include Pluronics and reverse Pluronics. Pluronics are a series of ABA block copolymers composed of poly(ethylene oxide)-poly(propylene oxide)-poly(ethylene oxide) blocks as generally represented in Formula VI:

HO(C₂H₄O)ₐ(C₃H₆O)_{b}(C₂H₄O)ₐH (VI)

wherein a is independently at least 1 and b is at least 1.

Reverse Pluronics are a series of BAB block copolymers, respectively composed of poly(propylene oxide)-poly(ethylene oxide)-poly(propylene oxide) blocks as generally represented in Formula VII:

HO(C₃H₆O)_{b}(C₂H₄O)ₐ(C₃H₆O)_{b}H (VII)

wherein a is at least 1 and b is independently at least 1. The poly(ethylene oxide), PEO, blocks are hydrophilic, whereas the poly(propylene oxide), PPO, blocks are hydrophobic in nature. The poloxamers in each series have varying ratios of PEO and PPO which ultimately determines the hydrophilic-lipophilic balance (HLB) of the material, i.e., the varying HLB values are based upon the varying values of a and b, a representing the number of hydrophilic poly(ethylene oxide) units (PEO) being present in the molecule and b representing the number of hydrophobic poly(propylene oxide) units (PPO) being present in the molecule.

Poloxamers and reverse poloxamers have terminal hydroxyl groups that can be terminal functionalized. An example of a terminal functionalized poloxamer and as discussed hereinbelow is poloxamer dimethacrylate (e.g., Pluronic® F127 dimethacrylate) as disclosed in U.S. Patent Application Publication No. 2003/0044468. Other examples include glycidyl-terminated copolymers of polyethylene glycol and polypropylene glycol as disclosed in U.S. Pat. No. 6,517,933.

Another example of a suitable polyether which can be end terminal functionalized is a poloxamine block copolymer. While the poloxamers and reverse poloxamers are considered to be difunctional molecules (based on the terminal hydroxyl groups), the poloxamines are in a tetrafunctional form, i.e., the molecules are tetrafunctional block copolymers terminating in primary hydroxyl groups and linked by a central diamine. One specific class of poloxamine block copolymers are those available under the trademark Tetronic (BASF). Poloxamines include Tetronic and reverse Tetronics. Poloxamines have the following general structure of Formula VIII:

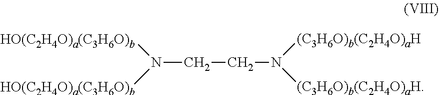

wherein a is independently at least 1 and b is independently at least 1.

The poloxamer and/or poloxamine is functionalized to provide the desired reactivity at the end terminal of the molecule. The functionality can be varied and is determined based upon the intended use of the functionalized PEO- and PPO-containing block copolymers. That is, the PEO- and PPO-containing block copolymers are reacted to provide end terminal functionality that is complementary with the intended device forming monomeric mixture. The term block copolymer as used herein shall be understood to mean a poloxamer and/or poloxamine as having two or more blocks in their polymeric backbone(s).

Generally, selection of the functional end group is determined by the functional group of the reactive molecule(s) in the mixture. For example, if the reactive molecule contains a carboxylic acid group, glycidyl methacrylate can provide a methacrylate end group. If the reactive molecule contains hydroxy or amino functionality, isocyanato ethyl methacrylate or (meth)acryloyl chloride can provide a methacrylate end group and vinyl chloro formate can provide a vinyl end group. A wide variety of suitable combinations of ethylenically unsaturated end groups and reactive molecules will be apparent to those of ordinary skill in the art. For example, the functional group may comprise a moiety selected from amine, hydrazine, hydrazide, thiol (nucleophilic groups), carboxylic acid, carboxylic ester, including imide ester, orthoester, carbonate, isocyanate, isothiocyanate, aldehyde, ketone, thione, alkenyl, acrylate, methacrylate, acrylamide, sulfone, maleimide, disulfide, iodo, epoxy, sulfonate, thiosulfonate, silane, alkoxysilane, halosilane, and phosphoramidate. More specific examples of these groups include succinimidyl ester or carbonate, imidazolyl ester or carbonate, benzotriazole ester or carbonate, p-nitrophenyl carbonate, vinyl sulfone, chloroethylsulfone, vinylpyridine, pyridyl disulfide, iodoacetamide, glyoxal, dione, mesylate, tosylate, and tresylate. Also included are other activated carboxylic acid derivatives, as well as hydrates or protected derivatives of any of the above moieties (e.g., aldehyde hydrate, hemiacetal, acetal, ketone hydrate, hemiketal, ketal, thioketal, thioacetal). Preferred electrophilic groups include succinimidyl carbonate, succinimidyl ester, maleimide, benzotriazole carbonate, glycidyl ether, imidazoyl ester, p-nitrophenyl carbonate, acrylate, tresylate, aldehyde, and orthopyridyl disulfide.

Representative examples of reaction sequences by which PEO- and PPO-containing block copolymers can be end-functionalized are provided below.

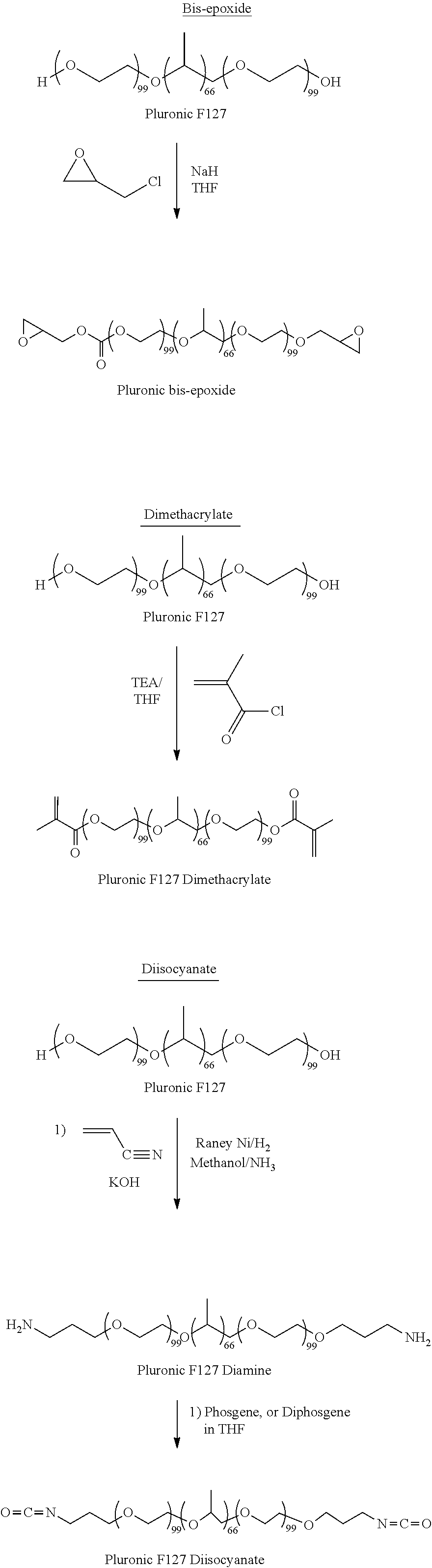

Further provided herein are certain exemplary, but non-limiting, examples of reactions for providing functionalized termini for PEO- and PPO-containing block copolymers. It is to be understood that one of ordinary skill in the art would be able to determine other reaction methods without engaging in an undue amount of experimentation. It should also be understood that any particular block copolymer molecule shown is only one chain length of a polydispersed population of the referenced material.

In an illustrative embodiment, the silicone biomedical device-forming mixture includes one or more of PEO- and PPO-containing block copolymers. An example of such a copolymer that can be used in monomeric mixture is Pluronic® F127, a block copolymer having the structure [(polyethylene oxide)₉₉-(polypropylene oxide)₆₆-(polyethylene oxide)₉₉]. The terminal hydroxyl groups of the copolymer are functionalized to allow for the reaction of the copolymer with other ophthalmic device forming monomers. Another example includes Pluronic 407 dimethacrylate having the following structure:

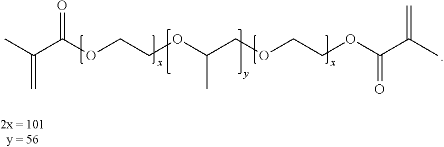

In an illustrative embodiment, an end terminal functionalized surfactant is selected from the group consisting of poloxamers having at least one end terminal functionalized, reverse poloxamers having at least one end terminal functionalized, poloxamines having at least one end terminal functionalized, reverse poloxamines having at least one end terminal functionalized and mixtures thereof.

In non-limiting illustrative embodiments, as may be combined with one or more of the preceding paragraphs, the surfactants can be present in the silicone biomedical device-forming mixture in an amount ranging from about 0.01 wt. % to about 20 wt. %, based on the total weight of the silicone biomedical device-forming mixture. In another illustrative embodiment, the surfactants can be present in the silicone biomedical device-forming mixture in an amount ranging from about 1 wt. % to about 10 wt. %, based on the total weight of the silicone biomedical device-forming mixture.

In non-limiting illustrative embodiments, as may be combined with one or more of the preceding paragraphs, the silicone biomedical device-forming mixture can further include a reactive (polymerizable) ultraviolet (UV) light absorber and/or a reactive blue-light absorber. Suitable reactive UV light absorbers can be any known reactive UV absorber. In non-limiting illustrative embodiments, suitable reactive UV light absorbers include, for example, 2-(2′-hydroxy-3′-methallyl-5′-methylphenyl)benzotriazole, commercially available as o-Methallyl Tinuvin P (“oMTP”) from Polysciences, Inc., Warrington, Pa., 3-(2H-benzo[d][1,2,3]triazol-2-yl)-4-hydroxyphenylethyl methacrylate, and 2-(3-(tert-butyl)-4-hydroxy-5-(5-methoxy-2H-benzo[d][1,2,3]triazol-2-yl)phenoxy)ethyl methacrylate.

In one illustrative embodiment, suitable UV light absorbers include, for example, one or more compounds of the following formulae:

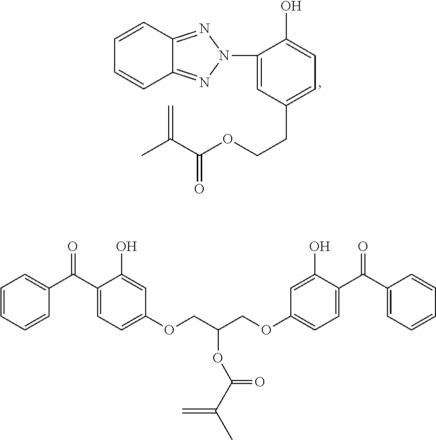 [SMILES: C=C(C)C(=O)OCCc1ccc(O)c(-n2nc3ccccc3n2)c1 ; C=C(C)C(=O)OC(COc1ccc(C(=O)c2ccccc2)c(O)c1)COc1ccc(C(=O)c2ccccc2)c(O)c1]

### (2-Propenoic acid, 2-methyl,2-(4-benzoyl-3-hydroxyphenoxy)-1-[(4-benzoyl3-hydroxyphenoxy)methyl ester),

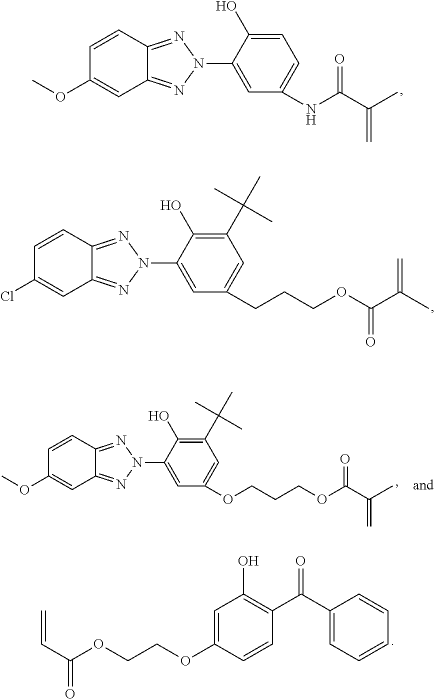

These compounds are merely illustrative and not intended to be limiting. Any known UV light absorber or later developed UV light absorber are contemplated for use herein.

In illustrative embodiments, the UV light absorbers can be present in the monomeric mixture in an amount ranging from about 0.1 wt. % to about 5 wt. %, based on the total weight of the monomeric mixture. In another illustrative embodiment, the UV light absorbers can be present in the monomeric mixture in an amount ranging from about 1.5 wt. % to about 2.5 wt. %, based on the total weight of the monomeric mixture. In yet another non-limiting illustrative embodiment, the UV light absorbers can be present in the monomeric mixture in an amount ranging from about 1.5 wt. % to about 2 wt. %, based on the total weight of the monomeric mixture.

Many reactive blue-light absorbing compounds are known. Preferred reactive blue-light absorbing compounds are those described in U.S. Pat. Nos. 5,470,932; 8,207,244; and 8,329,775, the contents of which are hereby incorporated by reference. In one embodiment, a blue-light absorbing dye is N-2-[3-(2′-methylphenylazo)-4-hydroxyphenyl]ethyl methacrylamide. In illustrative embodiments, the blue-light absorbers can be present in the monomeric mixture in an amount ranging from about 0.005 to about 1 wt. %, based on the total weight of the monomeric mixture. In another illustrative embodiment, the blue-light absorbers can be present in the monomeric mixture in an amount ranging from about 0.01 wt. % to about 1 wt. %, based on the total weight of the monomeric mixture.

The silicone biomedical device-forming mixtures disclosed herein may further contain, as necessary and within limits not to impair the purpose and effect of the illustrative embodiments disclosed herein, various additives such as an antioxidant, coloring agent, toughening agents and the like and other constituents as is well known in the art.

The silicone biomedical devices of the illustrative embodiments, e.g., contact lenses or intraocular lenses, can be prepared by polymerizing the foregoing silicone biomedical device-forming mixtures to form a product that can be subsequently formed into the appropriate shape by, for example, lathing, injection molding, compression molding, cutting and the like. For example, in producing contact lenses, the initial mixture may be polymerized in tubes to provide rod-shaped articles, which are then cut into buttons. The buttons may then be lathed into contact lenses.

Alternately, the silicone biomedical devices such as contact lenses may be cast directly in molds, e.g., polypropylene molds, from the mixtures, e.g., by spincasting and static casting methods. Spincasting methods are disclosed in U.S. Pat. Nos. 3,408,429 and 3,660,545, and static casting methods are disclosed in U.S. Pat. Nos. 4,113,224, 4,197,266, and 5,271,875. Spincasting methods involve charging the mixtures to be polymerized to a mold, and spinning the mold in a controlled manner while exposing the mixture to a radiation source such as UV light. Static casting methods involve charging the monomeric mixture between two mold sections, one mold section shaped to form the anterior lens surface and the other mold section shaped to form the posterior lens surface, and curing the mixture while retained in the mold assembly to form a lens, for example, by free radical polymerization of the mixture.

Examples of free radical reaction techniques to cure the lens material include thermal radiation, infrared radiation, electron beam radiation, gamma radiation, ultraviolet (UV) radiation, and the like; or combinations of such techniques may be used. U.S. Pat. No. 5,271,875 describes a static cast molding method that permits molding of a finished lens in a mold cavity defined by a posterior mold and an anterior mold. As an additional method, U.S. Pat. No. 4,555,732 discloses a process where an excess of a mixture is cured by spincasting in a mold to form a shaped article having an anterior lens surface and a relatively large thickness, and the posterior surface of the cured spincast article is subsequently lathed to provide a contact lens having the desired thickness and posterior lens surface.

Polymerization may be facilitated by exposing the mixture to heat and/or radiation, such as ultraviolet light, visible light, or high energy radiation. A polymerization initiator may be included in the mixture to facilitate the polymerization step. Representative examples of free radical thermal polymerization initiators include organic peroxides such as acetyl peroxide, lauroyl peroxide, decanoyl peroxide, stearoyl peroxide, benzoyl peroxide, tertiarylbutyl peroxypivalate, peroxydicarbonate, and the like. Representative UV initiators are those known in the art and include benzoin methyl ether, benzoin ethyl ether, Darocure® 1173, 1164, 2273, 1116, 2959, 3331 (EM Industries) and Irgacure® 651, 184 and 2959 (Ciba-Geigy), 2,2′Azobis(2-methylpropionitrile) (VAZO 64) and the like. Generally, the initiator will be employed in the mixture at a concentration of about 0.01 to about 5 percent by weight of the total mixture.

Polymerization is generally performed in a reaction medium, such as, for example, a solution or dispersion using a solvent, e.g., water or an alkanol containing from 1 to 4 carbon atoms such as methanol, ethanol or propan-2-ol. Alternatively, a mixture of any of the above solvents may be used.

Generally, polymerization can be carried out for about 15 minutes to about 72 hours, and under an inert atmosphere of, for example, nitrogen or argon. If desired, the resulting polymerization product can be dried under vacuum, e.g., for about 5 to about 72 hours or left in an aqueous solution prior to use.

Polymerization of the silicone biomedical device-forming mixtures will yield a polymer, that when hydrated, preferably forms a hydrogel. When producing a hydrogel lens, the silicone biomedical device-forming mixture may further include at least a diluent that is ultimately replaced with water when the polymerization product is hydrated to form a hydrogel. The amount of diluent used should be less than about 50 wt. %, and in most cases, the diluent content will be less than about 30 wt. %. However, in a particular polymer system, the actual limit will be dictated by the solubility of the various monomers in the diluent. In order to produce an optically clear copolymer, it is important that a phase separation leading to visual opacity does not occur between the comonomers and the diluent, or the diluent and the final copolymer.

Furthermore, the maximum amount of diluent which may be used will depend on the amount of swelling the diluent causes the final polymers. Excessive swelling will or may cause the copolymer to collapse when the diluent is replaced with water upon hydration. Suitable diluents include, but are not limited to, ethylene glycol, glycerine, liquid poly(ethylene glycol), alcohols, alcohol/water mixtures, ethylene oxide/propylene oxide block copolymers, low molecular weight linear poly(2-hydroxyethyl methacrylate), glycol esters of lactic acid, formamides, ketones, dialkylsulfoxides, butyl carbitol, and the like and mixtures thereof.

If necessary, it may be desirable to remove residual diluent from the lens before edge-finishing operations which can be accomplished by evaporation at or near ambient pressure or under vacuum. An elevated temperature can be employed to shorten the time necessary to evaporate the diluent. The time, temperature and pressure conditions for the solvent removal step will vary depending on such factors as the volatility of the diluent and the specific monomeric components, as can be readily determined by one skilled in the art. If desired, the mixture used to produce the hydrogel lens may further include crosslinking and wetting agents known in the prior art for making hydrogel materials.

In the case of intraocular lenses, the silicone biomedical device-forming mixtures to be polymerized may further include a monomer for increasing the refractive index of the resultant polymerized product. Examples of such monomers include aromatic (meth)acrylates, such as phenyl (meth)acrylate, 2-phenylethyl (meth)acrylate, 2-phenoxyethyl methacrylate, and benzyl (meth)acrylate.

The silicone biomedical devices such as contact lenses obtained herein may be subjected to optional machining operations. For example, the optional machining steps may include buffing or polishing a lens edge and/or surface. Generally, such machining processes may be performed before or after the product is released from a mold part, e.g., the lens is dry released from the mold by employing vacuum tweezers to lift the lens from the mold, after which the lens is transferred by means of mechanical tweezers to a second set of vacuum tweezers and placed against a rotating surface to smooth the surface or edges. The lens may then be turned over in order to machine the other side of the lens.

The lens may then be transferred to individual lens packages containing a buffered saline solution. The saline solution may be added to the package either before or after transfer of the lens. Appropriate packaging designs and materials are known in the art. A plastic package is releasably sealed with a film. Suitable sealing films are known in the art and include foils, polymer films and mixtures thereof. The sealed packages containing the lenses are then sterilized to ensure a sterile product. Suitable sterilization means and conditions are known in the art and include, for example, autoclaving.

As one skilled in the art will readily appreciate other steps may be included in the molding and packaging process described above. Such other steps can include, for example, coating the formed lens, surface treating the lens during formation (e.g., via mold transfer), inspecting the lens, discarding defective lenses, cleaning the mold halves, reusing the mold halves, and the like and combinations thereof.

The following examples are provided to enable one skilled in the art to practice the invention and are merely illustrative. The examples should not be read as limiting the scope of the invention as defined in the claims.

Various polymerization products were formed as discussed below and characterized by standard testing procedures such as:

Water %: Two sets of six hydrated lenses or films are blotted dry on a piece of filter paper to remove excess water, and samples are weighed (wet weight). Samples are then placed in a microwave oven for 10 minutes inside a jar containing desiccant. The samples are then allowed to sit for 30 minutes to equilibrate to room temperature and reweighed (dry weight). The percent water is calculated from the wet and dry weights.

Contact Angle: Captive bubble contact angle data was collected on a First Ten Angstroms FTA-1000 prop Shape Instrument. All samples were rinsed in HPLC grade water prior to analysis in order to remove components of the packaging solution from the sample surface. Prior to data collection the surface tension of the water used for all experiments was measured using the pendant drop method. In order for the water to qualify as appropriate for use, a surface tension value of 70 to 72 dynes/cm was expected. All lens samples were placed onto a curved sample holder and submerged into a quartz cell filled with HPLC grade water. Advancing and receding captive bubble contact angles were collected for each sample. The advancing contact angle is defined as the angle measured in water as the air bubble is retracting from the lens surface (water is advancing across the surface). All captive bubble data was collected using a high-speed digital camera focused onto the sample/air bubble interface. The contact angle was calculated at the digital frame just prior to contact line movement across the sample/air bubble interface. The receding contact angle is defined as the angle measured in water as the air bubble is expanding across the sample surface (water is receding from the surface).

Modulus (g/mm²) was measured per ASTM 1708 employing an Instron (Model 4502) instrument where the film sample was immersed in borate buffered saline; an appropriate size of the film sample was gauge length 22 mm and width 4.75 mm, where the sample further has ends forming a dogbone shape to accommodate gripping of the sample with clamps of the Instron instrument, and a thickness of 100±50 microns.

In the examples, the following abbreviations are used.
NVP: N-vinyl-2-pyrrolidone.
TBE: 4-t-butyl-2-hydroxycyclohexyl methacrylate.
DMA: N,N-dimethylacrylamide
EGDMA: Ethylene glycol dimethacrylate
AMA: Allyl methacrylate.
HA: Hyaluronic acid.
HA-MA: Methacrylated hyaluronic acid represented by the following structure:

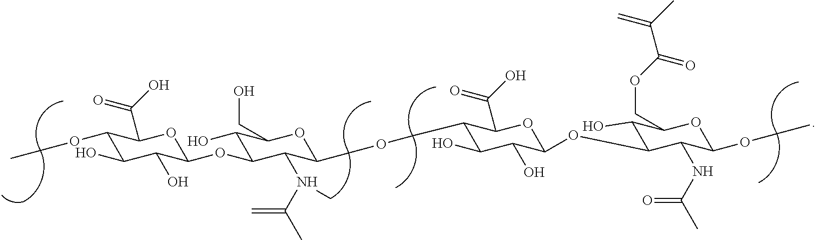 [SMILES: C=C(C)C(=O)OCC1OC(OCC)C(NC(C)=O)C(OC2OC(C(=O)O)C(COCC3OC(CO)C(O)C(OC4OC(C(=O)O)C(OCC)C(O)C4O)C3N(C)C(=C)C)C(O)C2O)C1O]

TrisMA: tris(trimethylsiloxy)silylpropyl methacrylate.

X-22-1666: N-[3-(9-butyl-1,1,3,3,5,5,7,7,9,9-decamethyl-1-pentasiloxanyl)propyl]-2-propenamide represented by the following structure:

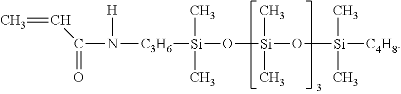

X-22-1666C: a silicone monomer represented by the following structure and available from ShinEtsu:

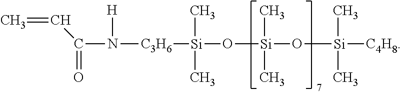

SIGMA: (3-methacryloxy-2-hydroxy propoxy)propyl bis(trimethyl siloxy)methyl silane having the following structure:

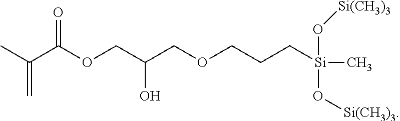

M1EDS6: a compound having the structure and available from Gelest:

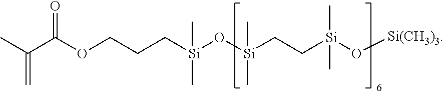

MCR-M11: a compound having the structure:

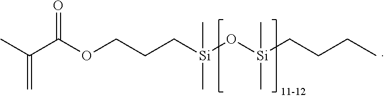

Vazo™ 64: azo bis-isobutylnitrile (AIBN).
Irgacure 2959: 2-Hydroxy-4′-(2-hydroxyethoxy)-2-methylpropiophenone,
PVP: Polyvinylpyrrolidone (K90),
UV416: 2-(4-Benzoyl-3-hydroxyphenoxy)ethyl acrylate.
Reactive blue 69: Disodium 1-amino-4-[[4-[(2-bromo-1-oxoallyl)amino]-2-sulphonatophenyl]amino]-9,10-dihydro-9,10-dioxoanthracene-2-sulphonate,
P407DM: Poloxamer 407 dimethacrylate having the following structure:

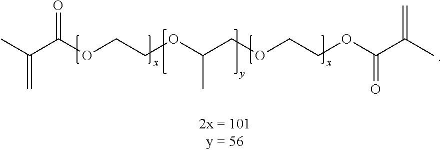

### Example 1

### Preparation of a HA-MA.

To a 1-Liter flask equipped with a stirbar was loaded with deionized (DI) water (490 mL). HA (10 g, Mₙ˜115 kDa) was dissolved in the stirring solution. A pH meter was immersed into the HA solution. Methacrylic anhydride (1.91 g, 12.4 mmol) was added into the solution at room temperature. The pH of the solution was maintained between 8.0 and 8.5 by adding an NaOH solution (20 wt. %) for 6 hours, then the solution was stirred overnight. The product was purified by dialysis (MWCO 6 to 8 kDa) against RO water for two days followed by lyophilized for two days. The product was redissolved in 300 mL of DI water and purified with dialysis and lyophilization again to afford a white powder of HA-MA with 10% of methacrylate.

### Example 2

### Preparation of X-22-1666 Having the Following Structure:

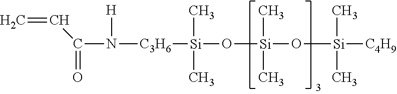

by the general reaction scheme.

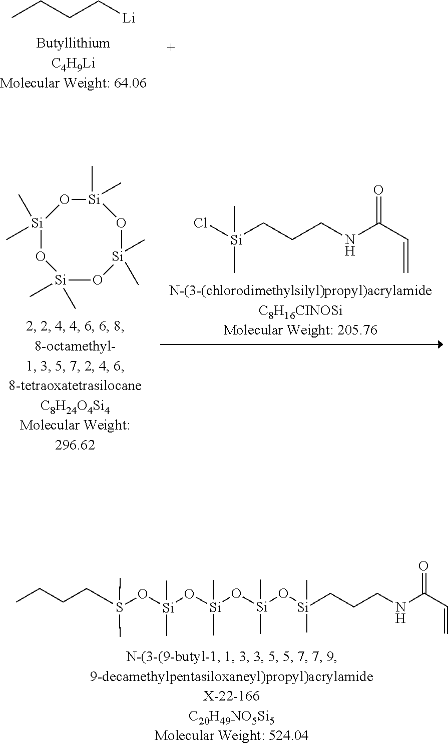

To an oven dried 2 L two-neck round bottom flask equipped with a magnetic stirring bar and condenser under N₂ atmosphere were added 2,2,4,4,6,6,8,8-octamethyl-1,3,5,7,2,4,6,8-tetraoxatetrasilocane (29.6 g g, 0.1 mol) and anhydrous cyclohexane (150 mL) under stirring in N₂ atmosphere. Butyllithium (6.4 g, 0.1 mol) was added to the above reaction mixture followed by the addition of cyclohexane (25 mL). After stirring for one hour, tertrahydrofuran (THF) (70 mL, distilled over sodium/benzophenone) was added and the reaction mixture continued to stir for 16 hours. Next, N-(3-(chlorodimethylsilyl)propyl)acrylamide (20.5 g, 0.1 mol) was added and the mixture was stirred for another 24 hours. The reaction mixture was then filtered and Silica gel (3.5 g, dried at 160° C. for 3 h) was added and the reaction mixture was stirred for an additional 4 hours. The reaction mixture was filtered through a bed of Celite (20 g) and butylated hydroxytoluene (BHT) (5 mg) was added to the filtrate. The filtrate was then concentrated under vacuum (40° C./0.3 mm Hg). Heptane (200 mL) was added to the concentrate with stirring and washed with DI water (100 mL), aqueous NaHCO₃ (2×100 mL, prepared by dissolving 10 g NaHCO₃ in 200 mL DI water), brine (100 mL) and finally DI water (100 mL). Heptane (50 mL) was added and dried over MgSO₄ (15 g) for 20 hours. The MgSO₄ was filtered off and the solvent was removed on rotary evaporator. The crude product was stirred over activated basic Alumina (30 g for 24 h) and then filtered over a thin bed of celite. Stripping off any residue solvent at 25° C. at 0.2 mmHg for 30 minutes yielded the desired product as clear oil in 40 g quantity.

### Examples 3-5 and Comparative Examples A-D

### Preparation of a Contact Lens.

Contact lenses were prepared using the reaction components listed in Table 2 below, as amounts per weight percent. The lenses were prepared by mixing the reaction components together in a scintillation vial while stirring or rolling for at least about 1 hour until all components were dissolved. The reaction mixture was then dispensed into a thermoplastic contact lens mold assembly and thermally cured at 90° C. for 2 hours after a purge with nitrogen to form a contact lens. The resulting lens were released from the mold, extracted with water for 2×3 minutes to remove residual monomers.

Once the lenses have been extracted, the lenses were placed into a vial or blister package filled with a buffered saline. The vials or blisters were sealed with a stopper or foil and autoclaved for about 30 minutes at about 121° C. The properties of the resulting contact lenses are also shown below in Table 3.

TABLE 2

| | Comp. | | | | Comp. | Comp. | Comp. |
|---|---|---|---|---|---|---|---|
| | Ex. A | Ex. 3 | Ex. 4 | Ex. 5 | Ex. B | Ex. C | Ex. D |
| Component | | | | | | | |
| (Parts) | | | | | | | |
| NVP | 63.00 | 63.00 | 63.00 | 63.00 | 63.00 | 63.00 | 63.00 |
| TBE | 6.00 | 6.00 | 6.00 | 6.00 | 6.00 | 6.00 | 6.00 |
| Propylene | 8.00 | 8.00 | 8.00 | 8.00 | 8.00 | 8.00 | 8.00 |
| Glycol | | | | | | | |
| DMA | 2.50 | 2.50 | 2.50 | 2.50 | 2.50 | 2.50 | 2.50 |
| EGDMA | 0.30 | 0.30 | 0.30 | 0.30 | 0.30 | 0.30 | 0.30 |
| AMA | 0.50 | 0.50 | 0.50 | 0.50 | 0.50 | 0.50 | 0.50 |
| 2% HA-MA | 10.00 | 10.00 | 10.00 | 10.00 | 10.00 | 10.00 | 10.00 |
| (in H₂O) | | | | | | | |
| UV-416 | 3.00 | 3.00 | 3.00 | 3.00 | 3.00 | 3.00 | 3.00 |
| X-22-1666 | — | 5.00 | — | — | — | — | — |
| Tris-MA | — | — | — | — | 5.00 | — | — |
| SiGMA | — | — | 5.00 | | — | | |
| X-22-1666C | | | | 5.00 | | | |
| (PDMS | | | | | | | |
| n = 7) | | | | | | | |
| MCR-M11 | — | — | — | — | — | 5.00 | — |
| M1EDS6 | — | — | — | — | — | — | 5.00 |
| P407DM | 5.00 | 5.00 | 5.00 | 5.00 | 5.00 | 5.00 | 5.00 |
| Vazo64 | 0.50 | 0.50 | 0.50 | 0.50 | 0.50 | 0.50 | 0.50 |
| Tint | 0.02 | 0.02 | 0.02 | 0.02 | 0.02 | 0.02 | 0.02 |

TABLE 3

| | | Properties | | | | | |
|---|---|---|---|---|---|---|---|
| | Comp. | | | | Comp. | Comp. | Comp. |
| | Ex. A | Ex. 3 | Ex. 4 | Ex. 5 | Ex. B | Ex. C | Ex. D |
| Clear/ | Clear | Clear | Clear | Clear | Cloudy | Cloudy | Cloudy |
| Cloudy | | | | | | | |
| Modulus | 49 | 51 | 62 | 53 | N/A | N/A | N/A |
| % WC | 77 | 75 | 75 | 74 | Opaque | Opaque | Opaque |
| Lens | 5/5 | 4/3 | 5/4 | 4/3 | lenses | lenses | lenses |
| Clarity/ | | | | | were not | were not | were not |
| Cloudy | | | | | tested | tested | tested |

The data in Table 3 shows that the contact lenses of Examples 3-5 prepared from a polymerization product of a mixture of a grafted glycosaminoglycan polymers comprising a glycosaminoglycan having a polymer backbone and one or more side chains comprising an ethylenically unsaturated reactive-containing residue grafted onto the polymer backbone, and a silicone biomedical device-forming monomers comprising at least one hydrogen donating group were optically clear, as compared to the contact lenses of Comparative Examples B-D prepared polymerization product of a mixture of a grafted glycosaminoglycan polymer comprising a glycosaminoglycan having a polymer backbone and one or more side chains comprising an ethylenically unsaturated reactive-containing residue grafted onto the polymer backbone and a silicone biomedical device-forming monomers containing no hydrogen donating groups (Comparative Examples B-D).

### Example 6 and Comparative Examples E-G

### Preparation of a Contact Lens.

Contact lenses were prepared using the reaction components listed in Table 4 below, as amounts per weight percent. First, 3-(trihydroxysilyl)propyl methacrylate was prepared by mixing 5% by weight of 3-(trimethoxysilyl)propyl methacrylate with 2% HA-MA in water and heated at 35° C. until the mixture turns clear. The reaction is a hydrolysis of the methoxy substituents to afford the 3-(trihydroxysilyl)propyl methacrylate. The mixture of water, HA-MA and 3-(trihydroxysilyl)propyl methacrylate was mixed directly into the monomer formulation.

The lenses were prepared by mixing the reaction components together in a scintillation vial while stirring or rolling for at least about 1 hour until all components were dissolved. The reaction mixture was then dispensed into a thermoplastic contact lens mold assembly and irradiated with UV light for 20 seconds to form a contact lens. The resulting lens were released from the mold, extracted with water for 3 minutes and placed into a buffered saline solution to remove residual monomers.

Once the lenses have been extracted the lenses were placed into a vial or blister package filled with a buffered saline. The vials or blisters were sealed with a stopper or foil and autoclaved for about 30 minutes at about 121° C. The properties of the resulting contact lenses are also shown below in Table 5.

TABLE 4

| Component (Wt. %) | Comp. Ex. E | Ex. 6 | Comp. Ex. F | Comp. Ex. G |
|---|---|---|---|---|
| Irgacure 2959 | 1.000 | 0.95 | 0.95 | 0.95 |
| P407 DM | 5.395 | 5.12525 | 5.12525 | 5.12525 |
| DMA | 58.600 | 55.67 | 55.67 | 55.67 |
| EGDMA | 1.200 | 1.14 | 1.14 | 1.14 |
| Reactive Blue 69 | 0.005 | 0.005 | 0.005 | 0.005 |
| 2% HA-MA | 26.000 | 24.7 | 24.7 | 24.7 |
| (in H₂O) | | | | |
| 10% PVP (in H₂O) | 7.800 | 7.41 | 7.41 | 7.41 |
| 3-(trihydroxysilyl)propyl methacrylate | — | 5.00 | | |
| 3-(trimethoxysilyl)propyl methacrylate | — | — | 5.00 | |
| TrisMA | — | — | | 5.00 |

TABLE 5

| | Properties | | | |
|---|---|---|---|---|
| | Comp. | | Comp. | Comp. |
| | Ex. E | Ex. 6 | Ex. F | Ex. G |
| Clear/Cloudy | Clear | Clear | — | — |
| Modulus | 45 | 60 | N/A | N/A |
| % WC | 83 | 75 | Monomer | Monomer |
| CBCA | 51 | 55 | and lenses | and lenses |
| Lens | 5/4 | 5/4 | were phase | were phase |
| Clarity/Cloudy | | | separated | separated |

The data in Table 5 shows that the contact lens of Example 6 prepared from a polymerization product of a mixture of a grafted glycosaminoglycan polymers comprising a glycosaminoglycan having a polymer backbone and one or more side chains comprising an ethylenically unsaturated reactive-containing residue grafted onto the polymer backbone, and a silicone biomedical device-forming monomers comprising at least one hydrogen donating group were optically clear, as compared to the contact lenses of Comparative Examples F-G prepared polymerization product of a mixture of a grafted glycosaminoglycan polymer comprising a glycosaminoglycan having a polymer backbone and one or more side chains comprising an ethylenically unsaturated reactive-containing residue grafted onto the polymer backbone and a silicone biomedical device-forming monomers containing no hydrogen donating groups (Comparative Examples F-G).

While compositions and methods are described in terms of “comprising” various components or steps, the compositions and methods can also “consist essentially of” or “consist of” the various components or steps, unless stated otherwise.

The terms “a,” “an,” and “the” are intended to include plural alternatives, e.g., at least one. The terms “including”, “with”, and “having”, as used herein, are defined as comprising (i.e., open language), unless specified otherwise.

Various numerical ranges are disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. For example, all numerical end points of ranges disclosed herein are approximate, unless excluded by proviso.

Values or ranges may be expressed herein as “about”, from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In another aspect, use of the term “about” means±20% of the stated value, ±15% of the stated value, ±10% of the stated value, ±5% of the stated value, ±3% of the stated value, or ±1% of the stated value.

Applicant reserves the right to proviso out or exclude any individual members of any such group of values or ranges, including any sub-ranges or combinations of sub-ranges within the group, that can be claimed according to a range or in any similar manner, if for any reason Applicant chooses to claim less than the full measure of the disclosure, for example, to account for a reference that Applicant may be unaware of at the time of the filing of the application. Further, Applicant reserves the right to proviso out or exclude any members of a claimed group.

According to one aspect of the present disclosure, a silicone biomedical device comprises a polymerization product of a biomedical device-forming mixture comprising (a) one or more grafted glycosaminoglycan polymers comprising a glycosaminoglycan having a polymer backbone and one or more side chains comprising an ethylenically unsaturated reactive-containing residue grafted onto the polymer backbone; and (b) one or more silicone biomedical device-forming monomers comprising at least one hydrogen donating group.

In non-limiting illustrative embodiments, as may be combined with one or more of the preceding paragraphs, the one or more grafted glycosaminoglycan polymers are selected from the group consisting of chondroitin, chondroitin sulfate, dermatan, dermatan sulfate, heparin, heparan sulfate, heparosan, hyaluronan, and hyaluronic acid or a salt thereof.

In non-limiting illustrative embodiments, as may be combined with one or more of the preceding paragraphs, the one or more grafted glycosaminoglycan polymers have a degree of grafting ranging from about 2% to about 30%.

In non-limiting illustrative embodiments, as may be combined with one or more of the preceding paragraphs, the ethylenically unsaturated reactive-containing residue is a (meth)acrylate-containing residue.

In non-limiting illustrative embodiments, as may be combined with one or more of the preceding paragraphs, the one or more grafted glycosaminoglycan polymers have a degree of methacrylation ranging from about 0.5% to about 50%.

In non-limiting illustrative embodiments, as may be combined with one or more of the preceding paragraphs, the glycosaminoglycan is hyaluronic acid or a salt thereof and the ethylenically unsaturated reactive-containing residue is a (meth)acrylate-containing residue.

In non-limiting illustrative embodiments, as may be combined with one or more of the preceding paragraphs, the at least one hydrogen donating group include one or more of hydroxy, amino, SH and carboxyl.

In non-limiting illustrative embodiments, as may be combined with one or more of the preceding paragraphs, the at least one hydrogen donating group is OH or NH.

In non-limiting illustrative embodiments, as may be combined with one or more of the preceding paragraphs, the one or more silicone biomedical device-forming monomers are one or more hydroxyl-functionalized silicone biomedical device-forming monomers.

In non-limiting illustrative embodiments, as may be combined with one or more of the preceding paragraphs, the one or more hydroxyl-functionalized silicone biomedical device-forming monomers include one or more monomers of Formulae I, II or III:

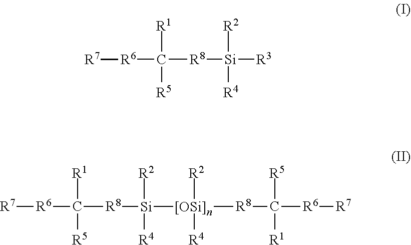

wherein:
n is an integer between 3 and 35, or between 4 and 25;
R¹ is hydrogen or C₁₋₆ alkyl;
R², R³, and R⁴, are independently a substituted or unsubstituted C₁₋₆ alkyl group, a C₁₋₆ triC₁₋₆ alkylsiloxy group, a phenyl group, a naphthyl group, a substituted phenyl group, and a substituted naphthyl group;
R⁵ is a hydroxyl group, an alkyl group containing one or more hydroxyl groups, or (CH₂(CR⁹R¹⁰)_{y}O)ₓ)—R¹¹ wherein y is 1 to 5, x is an integer of 1 to 100;
R⁶ is a divalent group comprising up to 20 carbon atoms;
R⁷ is a free radical reactive group;
R⁸ is a divalent or trivalent group comprising up to 20 carbon atoms; and
R⁹-R¹¹ are independently hydrogen, an alkyl group having up to 10 carbon atoms optionally substituted with at least one polar functional group, or:

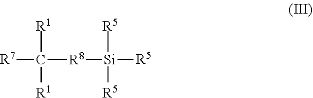

wherein R¹, R⁵, R⁷ and R⁸ have the aforestated meanings.

In non-limiting illustrative embodiments, as may be combined with one or more of the preceding paragraphs, the hydroxyl-functionalized silicone biomedical device-forming monomers include a monomer represented by the structure:

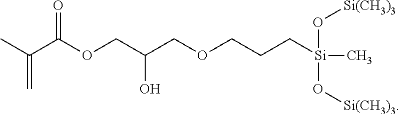

In non-limiting illustrative embodiments, as may be combined with one or more of the preceding paragraphs, the one or more silicone biomedical device-forming monomers are one or more amino-functionalized silicone biomedical device-forming monomers.

In non-limiting illustrative embodiments, as may be combined with one or more of the preceding paragraphs, the one or more amino-functionalized silicone biomedical device-forming monomers are represented by a structure of Formula IV:

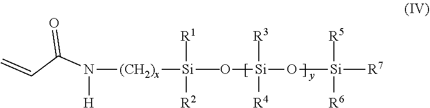

wherein R¹, R², R³ and R⁴ are independently hydrogen, an alkyl group, a halo alkyl group, a cycloalkyl group, a heterocycloalkyl group, an alkenyl group, a haloalkenyl group, an aryl group and a heteroaryl group; R⁵, R⁶ and R⁷ are independently a straight or branched alkyl group; x is from 1 to 6; and y is from 3 to 7.

In non-limiting illustrative embodiments, as may be combined with one or more of the preceding paragraphs, the one or more silicone biomedical device-forming monomers further comprise at least one ethylenically unsaturated reactive group.

In non-limiting illustrative embodiments, as may be combined with one or more of the preceding paragraphs, the one or more silicone biomedical device-forming monomers have a ratio of silicon atoms to hydrogen donating groups of from about 1:1 to about 9:1.

In non-limiting illustrative embodiments, as may be combined with one or more of the preceding paragraphs, the one or more silicone biomedical device-forming monomers have a ratio of silicon atoms to hydrogen donating groups of from about 1:1 to about 4:1.

In non-limiting illustrative embodiments, as may be combined with one or more of the preceding paragraphs, the one or more silicone biomedical device-forming monomers have a ratio of silicon atoms to hydrogen donating groups of from about 1:1 to about 1:5.

In non-limiting illustrative embodiments, as may be combined with one or more of the preceding paragraphs, the silicone biomedical device-forming mixture comprises:
about 0.1 to about 1.0 wt. %, based on the total weight of the silicone biomedical device-forming mixture, of the one or more grafted glycosaminoglycan polymers; and
about 1 to about 30 wt. %, based on the total weight of the silicone biomedical device-forming mixture, of the one or more silicone biomedical device-forming monomers.

In non-limiting illustrative embodiments, as may be combined with one or more of the preceding paragraphs, the silicone biomedical device-forming mixture further comprises one or more non-silicone biomedical device-forming monomers.

In non-limiting illustrative embodiments, as may be combined with one or more of the preceding paragraphs, the one or more non-silicone biomedical device-forming monomers are selected from the group consisting of an unsaturated carboxylic acid, an acrylamide, a vinyl lactam, a poly(alkyleneoxy)(meth)acrylate, (meth)acrylic acid, a hydroxyl-containing-(meth)acrylate, a hydrophilic vinyl carbonate, a hydrophilic vinyl carbamate monomer, a hydrophilic oxazolone monomer, and mixtures thereof.

In non-limiting illustrative embodiments, as may be combined with one or more of the preceding paragraphs, the silicone biomedical device-forming mixture comprises about 30 wt. % to about 90 wt. %, based on the total weight of the silicone biomedical device-forming mixture, of the one or more non-silicone biomedical device-forming monomers.

In non-limiting illustrative embodiments, as may be combined with one or more of the preceding paragraphs, the silicone biomedical device-forming mixture further comprises one or more crosslinking agents.

In non-limiting illustrative embodiments, as may be combined with one or more of the preceding paragraphs, the silicone biomedical device-forming mixture further comprises one or more components selected from the group consisting of a wetting agent, a surfactant and an ultraviolet (UV) blocker.

In non-limiting illustrative embodiments, as may be combined with one or more of the preceding paragraphs, the silicone biomedical device has a water content of at least about 70 wt. %.

In non-limiting illustrative embodiments, as may be combined with one or more of the preceding paragraphs, the silicone biomedical device is optically clear.

In non-limiting illustrative embodiments, as may be combined with one or more of the preceding paragraphs, the silicone biomedical device is one of a contact lens, an intraocular lens and a hydrogel.

According to another aspect of the present disclosure, a method for making a silicone biomedical device comprises:
(a) providing a silicone biomedical device-forming mixture comprising (i) one or more grafted glycosaminoglycan polymers comprising a glycosaminoglycan having a polymer backbone and one or more side chains comprising an ethylenically unsaturated reactive-containing residue grafted onto the polymer backbone, and (ii) one or more silicone biomedical device-forming monomers comprising at least one hydrogen donating group;
(b) subjecting the mixture to polymerization conditions to provide a polymerized silicone biomedical device; and
(c) hydrating the polymerized silicone biomedical device.

In non-limiting illustrative embodiments, as may be combined with one or more of the preceding paragraphs, the one or more grafted glycosaminoglycan polymers are selected from the group consisting of chondroitin, chondroitin sulfate, dermatan, dermatan sulfate, heparin, heparan sulfate, heparosan, hyaluronan, and hyaluronic acid or a salt thereof.

In non-limiting illustrative embodiments, as may be combined with one or more of the preceding paragraphs, the one or more grafted glycosaminoglycan polymers have a degree of grafting ranging from about 2% to about 30%.

In non-limiting illustrative embodiments, as may be combined with one or more of the preceding paragraphs, the ethylenically unsaturated reactive-containing residue is a (meth)acrylate-containing residue.

In non-limiting illustrative embodiments, as may be combined with one or more of the preceding paragraphs, the one or more grafted glycosaminoglycan polymers have a degree of methacrylation ranging from about 0.5% to about 50%.

In non-limiting illustrative embodiments, as may be combined with one or more of the preceding paragraphs, the glycosaminoglycan is hyaluronic acid or a salt thereof and the ethylenically unsaturated reactive-containing residue is a (meth)acrylate-containing residue.

In non-limiting illustrative embodiments, as may be combined with one or more of the preceding paragraphs, the at least one hydrogen donating group include one or more of hydroxy, amino, SH and carboxyl.

In non-limiting illustrative embodiments, as may be combined with one or more of the preceding paragraphs, the at least one hydrogen donating group is OH or NH.

In non-limiting illustrative embodiments, as may be combined with one or more of the preceding paragraphs, the one or more silicone biomedical device-forming monomers are one or more hydroxyl-functionalized silicone biomedical device-forming monomers.

In non-limiting illustrative embodiments, as may be combined with one or more of the preceding paragraphs, the one or more hydroxyl-functionalized silicone biomedical device-forming monomers include one or more monomers of Formulae I, II and III:

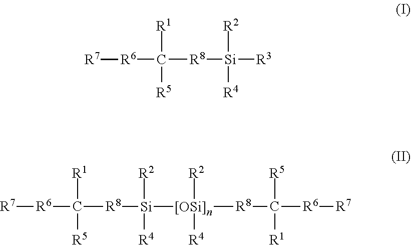

wherein:
n is an integer between 3 and 35, or between 4 and 25;
R¹ is hydrogen or C₁₋₆ alkyl;
R², R³, and R⁴, are independently a substituted or unsubstituted C₁₋₆ alkyl group, a C₁₋₆ triC₁₋₆ alkylsiloxy group, a phenyl group, a naphthyl group, a substituted phenyl group, and a substituted naphthyl group;
R⁵ is a hydroxyl group, an alkyl group containing one or more hydroxyl groups, or (CH₂(CR⁹R¹⁰)_{y}O)ₓ)—R¹¹ wherein y is 1 to 5, x is an integer of 1 to 100;
R⁶ is a divalent group comprising up to 20 carbon atoms;
R⁷ is a free radical reactive group;
R⁸ is a divalent or trivalent group comprising up to 20 carbon atoms; and
R⁹-R¹¹ are independently hydrogen, an alkyl group having up to 10 carbon atoms optionally substituted with at least one polar functional group, or:

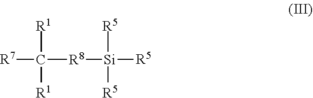

wherein R¹, R⁵, R⁷ and R⁸ have the aforestated meanings.

In non-limiting illustrative embodiments, as may be combined with one or more of the preceding paragraphs, the one or more hydroxyl-functionalized silicone biomedical device-forming monomers include a monomer represented by the structure:

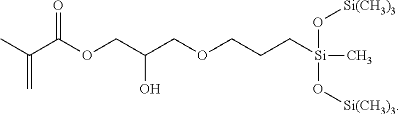

In non-limiting illustrative embodiments, as may be combined with one or more of the preceding paragraphs, the one or more silicone biomedical device-forming monomers are one or more amino-functionalized silicone biomedical device-forming monomers.

In non-limiting illustrative embodiments, as may be combined with one or more of the preceding paragraphs, the one or more amino-functionalized silicone biomedical device-forming monomers are represented by a structure of Formula IV:

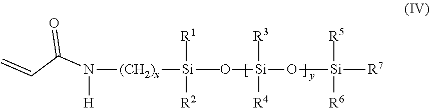

wherein R¹, R², R³ and R⁴ are independently hydrogen, an alkyl group, a halo alkyl group, a cycloalkyl group, a heterocycloalkyl group, an alkenyl group, a haloalkenyl group, an aryl group and a heteroaryl group; R⁵, R⁶ and R⁷ are independently a straight or branched alkyl group; x is from 1 to 6; and y is from 3 to 15.

In non-limiting illustrative embodiments, as may be combined with one or more of the preceding paragraphs, the one or more silicone biomedical device-forming monomers further comprise at least one ethylenically unsaturated reactive group.

In non-limiting illustrative embodiments, as may be combined with one or more of the preceding paragraphs, the one or more silicone biomedical device-forming monomers have a ratio of silicon atoms to hydrogen donating groups of from about 1:1 to about 9:1.

In non-limiting illustrative embodiments, as may be combined with one or more of the preceding paragraphs, the one or more silicone biomedical device-forming monomers have a ratio of silicon atoms to hydrogen donating groups of from about 1:1 to about 4:1.

In non-limiting illustrative embodiments, as may be combined with one or more of the preceding paragraphs, the one or more silicone biomedical device-forming monomers have a ratio of silicon atoms to hydrogen donating groups of from about 1:1 to about 1:5.

In non-limiting illustrative embodiments, as may be combined with one or more of the preceding paragraphs, the silicone biomedical device-forming mixture comprises:
about 0.1 to about 1.0 wt. %, based on the total weight of the silicone biomedical device-forming mixture, of the one or more grafted glycosaminoglycan polymers; and
about 1 to about 30 wt. %, based on the total weight of the silicone biomedical device-forming mixture, of the one or more silicone biomedical device-forming monomers.

In non-limiting illustrative embodiments, as may be combined with one or more of the preceding paragraphs, the silicone biomedical device-forming mixture further comprises one or more non-silicone biomedical device-forming monomers.

In non-limiting illustrative embodiments, as may be combined with one or more of the preceding paragraphs, the one or more non-silicone biomedical device-forming monomers are selected from the group consisting of an unsaturated carboxylic acid, an acrylamide, a vinyl lactam, a poly(alkyleneoxy)(meth)acrylate, (meth)acrylic acid, a hydroxyl-containing-(meth)acrylate, a hydrophilic vinyl carbonate, a hydrophilic vinyl carbamate monomer, a hydrophilic oxazolone monomer, and mixtures thereof.

In non-limiting illustrative embodiments, as may be combined with one or more of the preceding paragraphs, the silicone biomedical device-forming mixture comprises about 30 wt. % to about 90 wt. %, based on the total weight of the silicone biomedical device-forming mixture, of the one or more non-silicone biomedical device-forming monomers.

In non-limiting illustrative embodiments, as may be combined with one or more of the preceding paragraphs, the silicone biomedical device-forming mixture further comprises one or more crosslinking agents.

In non-limiting illustrative embodiments, as may be combined with one or more of the preceding paragraphs, the silicone biomedical device-forming mixture further comprises one or more components selected from the group consisting of a wetting agent, a surfactant and an ultraviolet (UV) blocker.

In non-limiting illustrative embodiments, as may be combined with one or more of the preceding paragraphs, the silicone biomedical device has a water content of at least about 70 wt. %.

In non-limiting illustrative embodiments, as may be combined with one or more of the preceding paragraphs, the silicone biomedical device is optically clear.

In non-limiting illustrative embodiments, as may be combined with one or more of the preceding paragraphs, the silicone biomedical device is one of a contact lens, an intraocular lens and a hydrogel.

Various features disclosed herein are, for brevity, described in the context of a single embodiment, but may also be provided separately or in any suitable sub-combination. All combinations of the embodiments are specifically embraced by the illustrative embodiments disclosed herein just as if each and every combination was individually and explicitly disclosed. In addition, all sub-combinations listed in the embodiments describing such variables are also specifically embraced by the present compositions and are disclosed herein just as if each and every such sub-combination was individually and explicitly disclosed herein.

It will be understood that various modifications may be made to the embodiments disclosed herein. Therefore, the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. A silicone biomedical device comprising a polymerization product of a biomedical device-forming mixture comprising:
(a) one or more grafted glycosaminoglycan polymers comprising a glycosaminoglycan having a polymer backbone and one or more side chains comprising an ethylenically unsaturated reactive-containing residue grafted onto the polymer backbone; and
(b) one or more silicone biomedical device-forming monomers comprising at least one hydrogen donating group.

2. The silicone biomedical device according to claim 1, wherein the one or more grafted glycosaminoglycan polymers are selected from the group consisting of chondroitin, chondroitin sulfate, dermatan, dermatan sulfate, heparin, heparan sulfate, heparosan, hyaluronan, and hyaluronic acid or a salt thereof.

3. The silicone biomedical device according to claim 1, wherein the one or more grafted glycosaminoglycan polymers have a degree of grafting ranging from about 2% to about 30%.

4. The silicone biomedical device according to claim 1, wherein the ethylenically unsaturated reactive-containing residue is a (meth)acrylate-containing residue.

5. The silicone biomedical device according to claim 4, wherein the one or more grafted glycosaminoglycan polymers have a degree of methacrylation ranging from about 0.5% to about 50%.

6. The silicone biomedical device according to claim 1, wherein the glycosaminoglycan is hyaluronic acid or a salt thereof and the ethylenically unsaturated reactive-containing residue is a (meth)acrylate-containing residue.

7. The silicone biomedical device according to claim 1, wherein the at least one hydrogen donating group include one or more of a hydroxy group, an amino group, a SH group and a carboxyl group.

8. The silicone biomedical device according to claim 1, wherein the at least one hydrogen donating group is OH or NH.

9. The silicone biomedical device according to claim 1, wherein the one or more silicone biomedical device-forming monomers comprise are one or more hydroxyl-functionalized silicone biomedical device-forming monomers.

10. The silicone biomedical device according to claim 9, wherein the one or more hydroxyl-functionalized silicone biomedical device-forming monomers comprise one or more monomers of Formulae I, II or III: 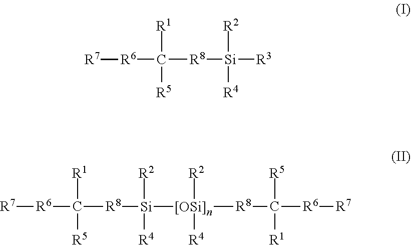
wherein:
n is an integer between 3 and 35, or between 4 and 25;
R¹ is hydrogen or C₁₋₆ alkyl;
R², R³, and R⁴, are independently a substituted or unsubstituted C₁₋₆ alkyl group, a C₁₋₆ triC₁₋₆ alkylsiloxy group, a phenyl group, a naphthyl group, a substituted phenyl group, and a substituted naphthyl group;
R⁵ is a hydroxyl group, an alkyl group containing one or more hydroxyl groups, or (CH₂(CR⁹R¹⁰)_{y}O)ₓ)—R¹¹ wherein y is 1 to 5, x is an integer of 1 to 100;
R⁶ is a divalent group comprising up to 20 carbon atoms;
R⁷ is a free radical reactive group;
R⁸ is a divalent or trivalent group comprising up to 20 carbon atoms; and
R⁹-R¹¹ are independently hydrogen, an alkyl group having up to 10 carbon atoms optionally substituted with at least one polar functional group, or: 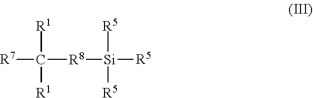
wherein R¹, R⁵, R⁷ and R⁸ have the aforestated meanings.

11. The silicone biomedical device according to claim 1, wherein the one or more silicone biomedical device-forming monomers comprise one or more amino-functionalized silicone biomedical device-forming monomers.

12. The silicone biomedical device according to claim 11, wherein the one or more amino-functionalized silicone biomedical device-forming monomers are represented by a structure of Formula IV: 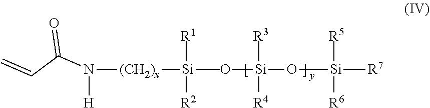
wherein R¹, R², R³ and R⁴ are independently hydrogen, an alkyl group, a halo alkyl group, a cycloalkyl group, a heterocycloalkyl group, an alkenyl group, a haloalkenyl group, an aryl group and a heteroaryl group; R⁵, R⁶ and R⁷ are independently a straight or branched alkyl group; x is from 1 to 6; and y is from 3 to 7.

13. The silicone biomedical device according to claim 1, wherein the one or more silicone biomedical device-forming monomers have a ratio of silicon atoms to hydrogen donating groups of from about 1:1 to about 9:1.

14. The silicone biomedical device according to claim 1, wherein the one or more silicone biomedical device-forming monomers have a ratio of silicon atoms to hydrogen donating groups of from about 1:1 to about 1:5.

15. The silicone biomedical device according to claim 1, wherein the silicone biomedical device-forming mixture comprises:
about 0.1 to about 1.0 wt. %, based on the total weight of the silicone biomedical device-forming mixture, of the one or more grafted glycosaminoglycan polymers; and
about 1 to about 30 wt. %, based on the total weight of the silicone biomedical device-forming mixture, of the one or more silicone biomedical device-forming monomers.

16. The silicone biomedical device according to claim 1, wherein the silicone biomedical device-forming mixture further comprises one or more non-silicone biomedical device-forming monomers.

17. The silicone biomedical device according to claim 1, having a water content of at least about 70 wt. %.

18. The silicone biomedical device according to claim 1, which is optically clear.

19. The silicone biomedical device according to claim 1, which is one of a silicone contact lens, a silicone intraocular lens or a silicone hydrogel.

20. A method for making a silicone biomedical device comprising:
(a) providing a silicone biomedical device-forming mixture comprising (i) one or more grafted glycosaminoglycan polymers comprising a glycosaminoglycan having a polymer backbone and one or more side chains comprising an ethylenically unsaturated reactive-containing residue grafted onto the polymer backbone, and (ii) one or more silicone biomedical device-forming monomers comprising at least one hydrogen donating group;
(b) subjecting the mixture to polymerization conditions to provide a polymerized silicone biomedical device; and
(c) hydrating the polymerized silicone biomedical device.
